(19) United States
(12) Patent Application Publication
(10) Pub. No.: US 2019/0051411 A1
(43) Pub. Date: Feb. 14, 2019

(54) DECISION MAKING PLATFORM

(71) Applicant: Real Time Medical Systems, LLC, Linthicum Heights, MD (US)

(72) Inventors: Christopher L. Miller, Mercer, PA (US); Cory Samuel McDowell, Cranberry Township, PA (US); James E. Shearon, Pittsburgh, PA (US)

(21) Appl. No.: 16/101,885

(22) Filed: Aug. 13, 2018

Related U.S. Application Data
(60) Provisional application No. 62/544,200, filed on Aug. 11, 2017.

(51) Int. Cl. G16H 50/20; G16H 10/60; G06F 3/0484

(52) U.S. Cl. CPC G16H 50/20; G06F 3/04842; G16H 10/60

(57) ABSTRACT

A decision making platform includes a data extraction circuit that interprets electronic medical records (EMRs), and a data conditioning circuit that determines an aggregated patient data set from the EMRs. The platform includes a patient readmission prediction circuit that determines a readmission risk for a patient, and a reporting circuit that provides a readmission response value. The platform further includes a clinical dashboard circuit that implements a transitions of care dashboard having: a user interface accessible to an external computing device, a time selection interface, and a display of at least a part of the readmission response value using a selection from the time selection interface.

### CLAIM OF PRIORITY

This patent application claims priority to U.S. Provisional Patent Application Ser. No. 62/544,200 (RTMS-0001-P01) filed on Aug. 11, 2017, entitled DECISION MAKING PLATFORM.

The entire contents of U.S. Provisional Patent Application Ser. No. 62/544,200 are hereby incorporated by reference in its entirety.

### BACKGROUND

### Field

This disclosure is related to patient management, and more particularly but not exclusively relates to managing outcomes for long-term care patients.

### Description of the Related Art

A healthcare facility must track the continuity of clinical care for patients during their stay, but also understand events in patients' prior care, before their transition to a healthcare facility. Facilities such as hospitals, nursing homes, extended care facilities, assisted living facilities and the like, often use different proprietary systems, such as electronic medical records (EMRs), charting tools, outcomes measures, and the like. Additionally, certain government mandated tools, submissions, ratings, surveys and regulatory requirements (which may differ by state or other criteria), provide a data infrastructure that is a vast amalgamation of data, and data exchange and reporting tools, many of which are incompatible or cannot fully interoperate. Healthcare facility managers using previously known systems suffer from a number of challenges. For example, healthcare facility managers must make sense of the data in this heterogeneous environment, get differing systems' data to correspond, and be able to longitudinally track patient and facility trends. Current options for such data and reporting integration, and the leveraging of clinical and financial-related data, are limited. Accordingly, many healthcare facilities using previously known systems suffer from high rate of reimbursement rejection, sub-optimal treatment for patients, and returns by patients to the healthcare facility after initial release.

### SUMMARY

An example system includes a decision making platform having a number of circuits structured to functionally execute certain operations of the system. The example system includes a data extraction circuit that interprets a number of electronic medical records (EMRs), a data conditioning circuit that determines an aggregated patient data set in response to the number of EMRs, a patient readmission prediction circuit that determines a readmission risk for a patient in response to the aggregated patient data set, a reporting circuit that provides a readmission response value in response to the readmission risk for the patient, and a clinical dashboard circuit that implements a transitions of care dashboard, the transitions of care dashboard including a user interface accessible to at least one external computing device. In certain embodiments, the example transitions of care dashboard includes a time selection interface, and readmission rates corresponding to at least a portion of the aggregated patient data set. In certain embodiments, the clinical dashboard circuit further displays the provided readmission response value on the transitions of care dashboard, and performs sorting, grouping, and/or filtering the transitions of care dashboard in response to a selection of the time selection interface.

Certain further aspects of an example system are described following, any one or more of which may be present in certain embodiments. An example system includes the transitions of care dashboard further including a patient selection interface, and where the clinical dashboard circuit further displays a number of readmission values in response to a patient selection of the patient selection interface. An example system includes the transitions of care dashboard further including a number of readmission scores corresponding to a number of readmission events, where the number of readmission scores include weighted scores.

Another example system includes a decision making platform having a number of circuits structured to functionally execute certain operations of the system. The example system includes a data extraction circuit that interprets a number of electronic medical records (EMRs), a data conditioning circuit that determines an aggregated patient data set in response to the number of EMRs, a patient readmission prediction circuit that determines a readmission risk for a patient in response to the aggregated patient data set, and a reporting circuit that provides a readmission response value in response to the readmission risk for the patient.

Certain further aspects of an example system are described following, any one or more of which may be present in certain embodiments. An example system includes the readmissions response value(s) being one or more of: a treatment recommendation value, a patient alert value, a treatment time value, an intervention recommendation value, a narrative description of the alert, category(ies) of the alert, and/or a readmission risk report corresponding to a patient. An example system includes a clinical dashboard circuit that implements a transitions of care dashboard, the transitions of care dashboard including a user interface accessible to at least one external computing device, and where the transitions of care dashboard further includes one or more of: a number of readmission scores corresponding to a number of readmission events, readmission rates corresponding to at least a portion of the aggregated patient data set, a number of diagnosis values corresponding to a number of readmission events, and/or a number of comorbidity values corresponding to a plurality of readmission events. An example system further includes a clinical dashboard circuit that implements a transitions of care dashboard, the transitions of care dashboard including a user interface accessible to at least one external computing device, and readmission rates corresponding to at least a portion of the aggregated patient data set. The example readmission rates include a 30-day readmission rate value, a monthly readmission rate value, a readmission rate value for a selected time period, and/or a number of readmission rates each corresponding to a selected patient grouping category. In certain embodiments, an example system includes the transitions of care dashboard having a time selection interface, where the clinical dashboard circuit performs one or more of sorting, grouping, and/or filtering the transitions of care dashboard in response to a selection of the time selection interface. In certain embodiments, an example system includes the transitions of care dashboard having a patient selection interface, where the clinical dashboard circuit displays a number of readmission values in response to a patient selection of the patient selection interface. An example system includes the number of readmission values being one or more of: active diagnoses corresponding to the patient selection, comorbidities corresponding to the patient selection, a readmission rate value corresponding to the patient selection, a readmission score value corresponding to the patient selection, and/or a patient alert value history corresponding to the patient selection. An example system further includes the clinical dashboard circuit implementing a transitions of care dashboard, the transitions of care dashboard including a user interface accessible to at least one external computing device, and a number of readmission scores corresponding to a number of readmission events, where the number of readmission scores include weighted scores. An example system further includes the clinical dashboard circuit weighting the number of readmission scores according to one or more of the following criteria: a number of patient alert values, a number of patient alert values corresponding to a predetermined time period, a length of stay description for the aggregated patient data set, a set of diagnoses corresponding to a portion of the aggregated patient data set, a set of comorbidities corresponding to a portion of the aggregated patient data set, and/or a time dependent value for any of the foregoing.

An example procedure includes an operation to interpret a number of electronic medical records (EMRs), an operation to determine an aggregated patient data set in response to the number of EMRs, an operation to determine a readmission risk for a patient in response to the aggregate patient data set, and an operation to provide a readmission response value in response to the readmission risk for the patient.

Certain further aspects of an example procedure are described following, any one or more of which may be present in certain embodiments. An example procedure includes an operation to implement a transitions of care dashboard, where the transitions of care dashboard includes a user interface accessible to at least one external computing device, and where the transitions of care dashboard further includes one or more of: a number of readmission scores corresponding to the number of readmission events, readmission rates corresponding to at least a portion of the aggregated patient data set, a number of diagnosis values corresponding to the number of readmission events, and/or a number of comorbidity values corresponding to the number of readmission events. An example procedure includes an operation to implement a transitions of care dashboard, where the transitions of care dashboard includes a user interface accessible to at least one external computing device, and readmission rates corresponding to at least a portion of the aggregated patient data set. The example readmission rates include one or more of: a 30-day readmission rate value, a monthly readmission rate value, a readmission rate value for a selected time period, and/or a number of readmission rates each corresponding to a selected patient grouping category. An example transitions of care dashboard further includes a time selection interface, where the example procedure further includes an operation to perform sorting, grouping, and/or filtering the transitions of care dashboard in response to a selection of the time selection interface. An example transitions of care dashboard further includes a patient selection interface, where the example procedure further includes an operation to display a number of readmission values in response to a patient selection of the patient selection interface. An example procedure includes readmission values that are one or more of: active diagnoses corresponding to the patient selection, comorbidities corresponding to the patient selection, readmission rate values corresponding to the patient selection, a readmission score value corresponding to the patient selection, and/or a patient alert value history corresponding to the patient selection. An example procedure includes an operation to implement a transitions of care dashboard, where the transitions of care dashboard further includes a user interface accessible to at least one external computing device, and a number of readmission scores corresponding to the number of readmission events, where the number of readmission scores include weighted scores. An example procedure further includes an operation to weight the number of readmission scores according to one or more of the following criteria: a number of patient alert values, a number of patient alert values corresponding to a predetermined time period, a length of stay description for the aggregated patient data set, a set of diagnoses corresponding to a portion of the aggregated patient data set, a set of comorbidities corresponding to a portion of the aggregated patient data set, and/or a time dependent value for any of the foregoing.

### BRIEF DESCRIPTION OF THE FIGURES

FIG. 1 is a schematic depiction of a Decision Making Platform.

FIG. 2 is a schematic flow description of certain aspects of a Decision Making Platform.

FIG. 3 is a schematic depiction of an example clinical dashboard.

FIG. 4 is a schematic depiction of an example financial dashboard.

FIG. 5 is a schematic depiction of an example transitions of care dashboard.

FIG. 6 is a schematic depiction of a clinical report.

FIG. 7 is a schematic depiction of a financial report.

FIG. 8 is a schematic depiction of a quality measures report.

FIG. 9 is a schematic depiction of a transition of care report.

FIG. 10 is a schematic depiction of a facility portal usage report.

FIG. 11 is a schematic depiction of a portal usage report.

FIG. 12 is a schematic depiction of a system including a decision making platform.

FIG. 13 is a schematic flow diagram of a procedure for providing a readmission response value for a patient.

FIG. 14 is a schematic depiction of another embodiment of a system including a decision making platform.

FIG. 15 is a schematic flow diagram of a procedure for providing a clinical alert value.

FIG. 16 is a schematic depiction of another embodiment of a system including a decision making platform.

FIG. 17 is a schematic flow diagram of a procedure for providing a patient admission value.

### DETAILED DESCRIPTION

Certain embodiments of the present disclosure provide longitudinal tracking, that enables reporting for the purposes of regulatory compliance, and allows facility managers, including but not limited to clinicians, such as nurses, operations managers, and/or CFO's that are responsible for tracking financial standing of a facility. Accordingly, platforms of the present disclosure may import data sources from a diverse set of remote sources, systems and formats, map and standardize such data so that it may be aggregated in a format that may be accessed, analyzed and updated in real-time—for example as the course of patient care progresses and/or new data points are produced. Platforms of the present disclosure may determine and report results, trends, and mandated summaries for facility managers to track performance, maintain quality of care, fiscal health of facilities, and/or maximize proper reimbursement.

Data-driven healthcare analytics are a keystone of any operational decision. Electronic medical records (EMR) systems contain an immense amount of data, and the challenge lies within the fact that the data is “locked” inside the EMR, inaccessible to a healthcare facility and its financial managers, clinicians, and operations staff. Even when the data may be accessed, turning the data into actionable insights to drive efficiency and compliance in, for example, a long-term care facility, is often nearly impossible using previously known systems, and given staffing and budget constraints. In embodiments, an example Decision Making Platform of the present disclosure may extract key clinical data from a plurality of EMR systems and process the data within an analytics engine to produce real-time clinical and financial insights. Improving clinical endpoints and increasing reimbursement rates are an ongoing challenge for many long-term care facilities. Operations managers often lack the insights to know where and when to implement changes to achieve financial and clinical improvement. An example Decision Making Platform of the present disclosure may facilitate institutional improvements, such as real-time activities of daily living (ADL) visibility, tracking which residents have room for improvement in ADL and/or clinical care, and assist in identifying discrepancies in care provided versus care documented. Due to expanded hospital readmission penalties, healthcare providers often lose significant amounts of Medicare payments due to poor tracking of readmissions data, readmissions outcomes, and/or sub-optimal care resulting in unnecessary readmissions. Use of example Decision Making Platforms of the present disclosure may allow long-term care providers to detect changes in a residents' medical conditions as they develop, empowering the clinical staff to intervene before the condition requires more extensive treatments and/or treatments likely to lead to a readmission. This proactive approach to healthcare may benefit users based at least in part by reducing hospital readmissions, adding more billable days per month, and/or increasing Medicare reimbursement (or reducing reimbursement reductions or penalties).

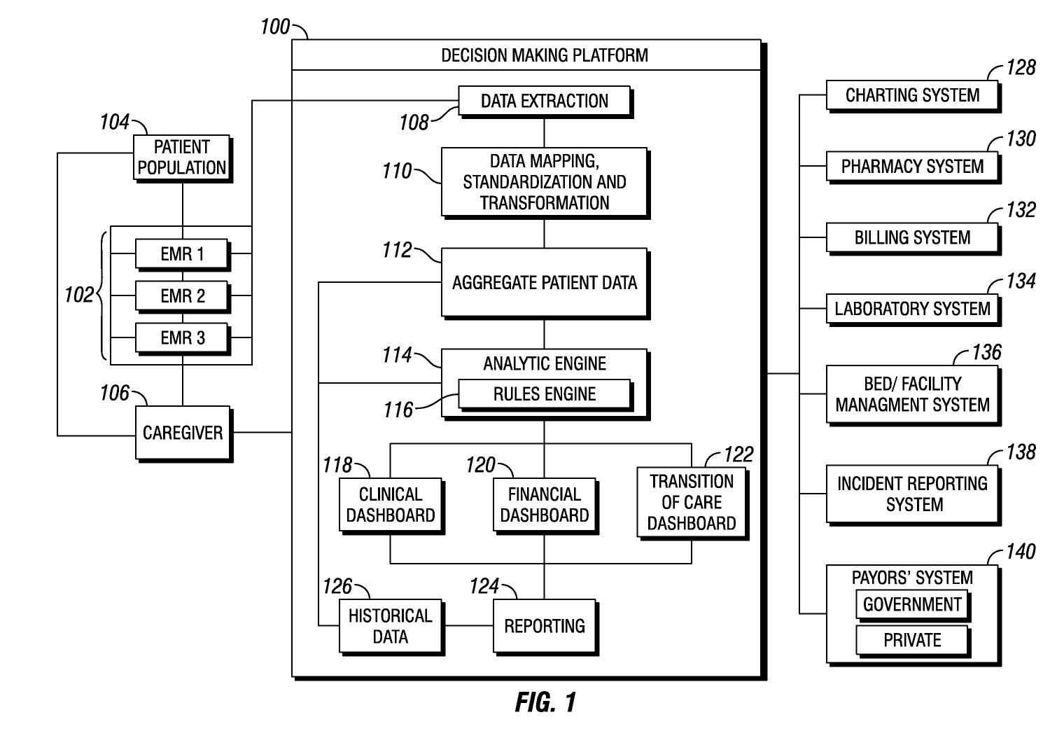

Currently available clinical analytics tools often require data and forms, for example the minimum data set (MDS), to be uploaded to a proprietary site, such as a website, where audited reports can only be prepared at a later time. Referencing FIG. 1, an example Decision Making Platform 100, as described herein, may receive data from a number of EMR systems 102 (e.g., EMR 1, EMR 2, EMR 3) in which health and other data relating to a patient population 104 is stored. The EMR systems 102 may be remote to the Decision Making Platform 100 and associated with healthcare facilities, including but not limited to hospitals, physicians' offices, outpatient clinics, emergency care facilities, surgical centers, specialty clinics, and the like. The Decision Making Platform 100 may also receive data directly from a caregiver 106, such as a nurse, physician, or other clinician or personnel associated with patient care. The Decision Making Platform 100 may include a data extraction module 108 capable of receiving data from the plurality of remote data sources (e.g., EMR system 102 and/or providers 106). The data from the remote data sources may be in a number of data formats, structures, interrelations, and codifications, which may be varied depending upon the data source and/or for a given data source. An example data mapping, standardization and transformation module 110 may create an aggregate patient data set 112 from the data, including performing such operations as standardizing data formats, data fields, data ranges, and other data characteristics. Accordingly, the aggregate patient data set 112 can be analyzed as a single, unified data set, even though the origin of the data is from a number of remote data sets which might not be interoperable in the originally communicated data formats or on the systems communicating the data. The example Decision Making Platform 100 includes an analytic engine 114 that may perform operations, calculations, modeling and the like on the aggregate patient data set 112, which may include applying rules using a rules engine 116, to present analytic summaries to users of the Decision Making Platform 100 utilizing any one or more of a number of user interfaces, such as a Clinical Dashboard(s) 118, Financial Dashboard(s) 120, Transition of Care Dashboard(s) 122, as described herein, or some other type of user interface. It will be understood that the description utilizing a clinical dashboard 118, a financial dashboard 120, and/or a transition of care dashboard 122 is provided for convenience of description, and any information provided to, or taken from, a user interface throughout the present disclosure may include any information, analysis, or other aspects described throughout the present disclosure. A given user interface may have any name or terminology, and may include portions of any one or more of the interfaces described throughout the present disclosure, including combinations and/or partial combinations of one or more interfaces described herein.

The example Decision Making Platform 100 includes a reporting module 124 may produce (and/or, access, receive, and/or display) data and analytic summaries, for example, relating to patients or residents of a long-term care facility. In certain embodiments, historical data 126 relating to patients and residents and their care may be stored in the Decision Making Platform 100 and/or stored in a location communicatively coupled (e.g., over a network, remote storage device, cloud storage, and/or over the internet) to the Decision Making Platform 100. The Decision Making Platform 100 may interoperate, and share data, analytics, reports or other information, with a plurality of remote data and computing facilities, including but not limited to charting systems 128, pharmacy systems 130, billing systems 132, laboratory systems 134, bed and/or facility management systems 136, incident reporting systems 138, and/or payors' systems 140 (e.g., government and/or private), or any other type of data or computing facility. In certain embodiments, any one or more of the modules or other aspects of the Decision Making Platform 100 may be remotely positioned from the Decision Making Platform 100, such as in communication over a network and/or the internet, and nevertheless form a portion of the schematic arrangement of the Decision Making Platform 100.

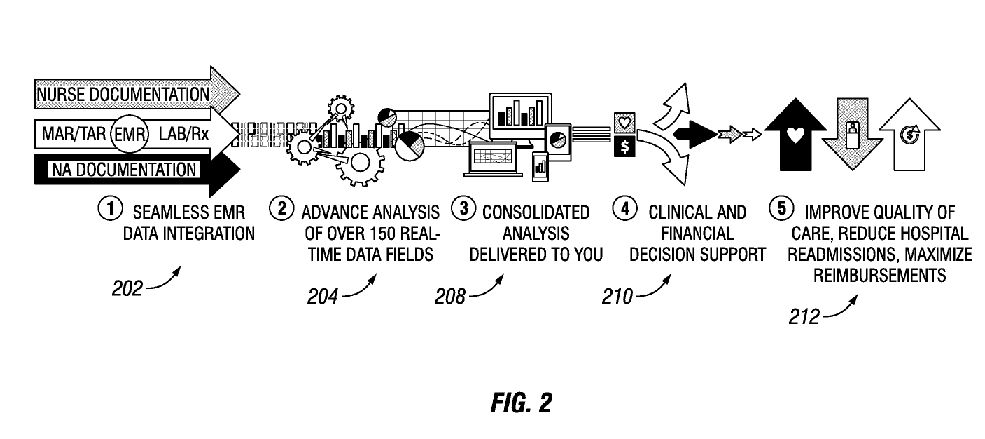

Referencing FIG. 2, a schematic flow description of certain operations of a Decision Making Platform 100, is depicted. The example flow description includes operations 202 to bring in data such as EMRs, nurse documentation, and/or lab results. The example flow description further includes operations 204 to analyze the data, including correcting, rationalizing, standardizing, and/or accommodating multiple input and/or multiple output formats and reporting protocols. Without limitation to any other aspects of the present disclosure, operation 204 further include determining patient readmission risks, long-term care facility scores, patient admission values, live quality measures, and/or clinical alert values in response to the operations 202. The example flow description further includes operations 206 to provide a consolidated analysis, including views of the data and views of analysis results determined at operation 204. In certain embodiments, operations 206 include providing one or more dashboards, reports, and/or communicating notifications and/or alerts. The example flow description further includes an operation 208 to provide clinical and/or financial support, for example ensuring that patient classifications are complete and correct, supporting resource planning and utilization of clinical care, providing alerts for clinical, financial, and/or administrative functions. The example flow description further includes operation 210 to improve quality of care for patients, reduce hospital readmission rates, and/or maximize reimbursements (or minimize reimbursement losses or penalties). Example operations 210 include providing timely treatment recommendation alerts and/or treatment change recommendations, highlighting patient quality changes that may not be visible to care providers (e.g., a slow decline in patient condition), determining comorbidities and/or other commonalities between patient groupings that may be causing readmissions and/or reimbursement losses, supporting multiple formats and/or recording protocols for input data such as EMRs, analyzing the data to determine where in the treatment system activities are occurring that cause loss of patient care quality and/or loss of reimbursement, and/or analyzing the data to determine where in the treatment system there are opportunities for a treatment, treatment change, and/or treatment adjustment to improve quality of care and/or reduce reimbursement losses.

In certain embodiments, operations of the Decision Making Platform 100 may not require any client intervention, as its methods and systems may utilize data straight from any EMR 102. This may benefit healthcare facilities and providers by providing a labor savings for a clinical team by eliminating the need to upload files. The data that the Decision Making Platform 100 utilizes from an EMR 102 may also be processed in real-time, making data summaries and analysis more actionable than data taken, for example, from an MDS or other data source that could be weeks old. Further, the Decision Making Platform 100 may utilize data from an EMR 102 multiple times per day, and for different uses, for example to ensure that the Dashboards (e.g., 118, 120, 122), as described herein, are current and meaningful to the client. Dashboards 118, 120, 122 may be made available via a web-portal that is associated with the Decision Making Platform 100, or may be sent to an email inbox immediately, as opposed to days or weeks later. Some current EMR systems may have a reporting capability that provides data for analysis, but those reports will need to be printed, consolidated, and audited, and they will be limited to that EMR system, and the associated EMRs 120, alone. Patients with data in other EMR systems must be analyzed separately. According to the methods and systems of the present disclosure, the Decision Making Platform 100 may perform these analytic and reporting processes for clinicians and managers and produce an actionable and interactive Dashboard 118, 120, 122 to keep the facility staff aware of, for example, clinical trends. The Decision Making Platform 100, in certain embodiments, also configures analysis, reports, and Dashboards 118, 120, 122, for the specific purposes of the particular client. This may positively impact clinical and financial endpoints and goals, as trends may be spotted and acted on in real-time instead of having to use, for example, MDS data that may be outdated. The real-time nature of the Decision Making Platform 100 may also facilitate improved error detection and reporting. As with any data entry process, human and/or automated error may be introduced, having a negative impact on reporting accuracy and negatively impacting the clinical or financial decisions that are made thereon. For example, an ADL error on one 14-day MDS may result in hundreds of dollars of lost reimbursement. The Decision Making Platform 100 may reduce and/or eliminate such errors with real-time, shift-by-shift data extracted from, for example, an EMR system. Such real-time information may be more relevant for meaningful clinical and financial decision making. For example, the Decision Making Platform 100 may positively impact resident care by providing clinical alerts and intervention recommendations to reduce adverse outcomes and return-to-acute (RTA) status for a patient, and/or reduction in rates of adverse outcomes or RTAs for a population or group of patients.

In embodiments of the present disclosure, the Decision Making Platform 100 may provide for enhanced data inputting, error detection, data cleaning and standardization, including but not limited to mapping data fields from, for example, one EMR system to data fields from a differing EMR system. This may enable standardizing the data ranges or other criteria for the purposes of accurate reporting and consistency with a healthcare facility's own data collection system. For example, current systems may use tools, such as MDS analytic tools, to cleanse MDS reports and may point out obvious documentation errors, but tools such as this don't increase substantive reporting accuracy or improve the long-term efficiency, financial viability, and/or patient outcomes of a facility. Basing clinical and financial decisions on dated MDS data may keep a facility in a reactionary state of responding to problems days or weeks after they arise. The Decision Making Platform 100, as described herein, may provide a proactive healthcare approach to, for example, skilled nursing facilities.

Analysis of the most accurate, up-to-date resident information taken from an EMR system, using the Decision Making Platform 100, may provide clinicians with real-time data to predict declines in resident health and recommend specific interventions to improve quality outcomes for each resident, without having to complete and submit an MDS, and/or before completing and submitting an MDS. One aspect of successful healthcare data analysis is how accurate and recent the data set is. Clinical decisions based on old, stagnant MDS data are just as outdated as the data itself, and are subject to being outdated relative to the present condition of the patient or treatment, and/or too late to implement early or preventative care.

In embodiments, the Decision Making Platform 100 may require no extra documentation, but rather utilize the data already being collected by clinicians as part of, for example, an EMR system. Reports may be delivered via email, or any other means, and printed or viewed on a mobile device, including but not limited to a smart phone, tablet, computer, or some other device type. In healthcare facilities, duplicate data entry often wastes clinicians' time, taking the focus away from providing care. Unlike other clinical assessment tools that forces clinicians to manually enter data into their system separately, the Decision Making Platform 100 does not require any duplicate documentation or data entry.

In embodiments, the Decision Making Platform 100 may ease the burden of printing and analyzing multiple electronic reports from, for example, a nursing staff by providing comprehensive clinical insights and intervention recommendations in one intuitive report and/or Dashboard 118, 120, 122, as described herein. This may have the added benefit of allowing nurses and/or other staff to spend more time at each resident's bedside providing essential care and reducing adverse outcomes. Reducing long-term care hospital readmissions, which falls largely on the shoulders of long-term care providers, may have the most significant impact on lowering healthcare costs in the United States, according to a 2014 ASQ survey of health quality experts. Using the real-time healthcare analytics enabled by the Decision Making Platform 100, long-term care providers may detect changes in a resident's medical condition as they develop, empowering the clinical staff to intervene before the condition requires more extensive treatments or readmission to the hospital. When a resident's data indicates the potential for a negative outcome based on predefined parameters, the facility may be presented with a clinical alert and specific intervention recommendations so assessment and treatment can be performed immediately. Additionally or alternatively, a Dashboard 118, 120, 122 and/or an alert can highlight the clinical alert and/or specific intervention recommendation, providing ready notification to the appropriate health care provider. While other systems analyze only Medicare or Managed Care residents, the Decision Making Platform 100 is payor-agnostic, analyzing all payors to give a healthcare facility a complete view of financial and clinical optimization opportunities. Readmitting residents to the hospital can be damaging to a long-term care facility. Not only does this introduce financial risk, but it may jeopardize the reputation of the facility in the community, among referring physicians and hospitals, and with potential customers.

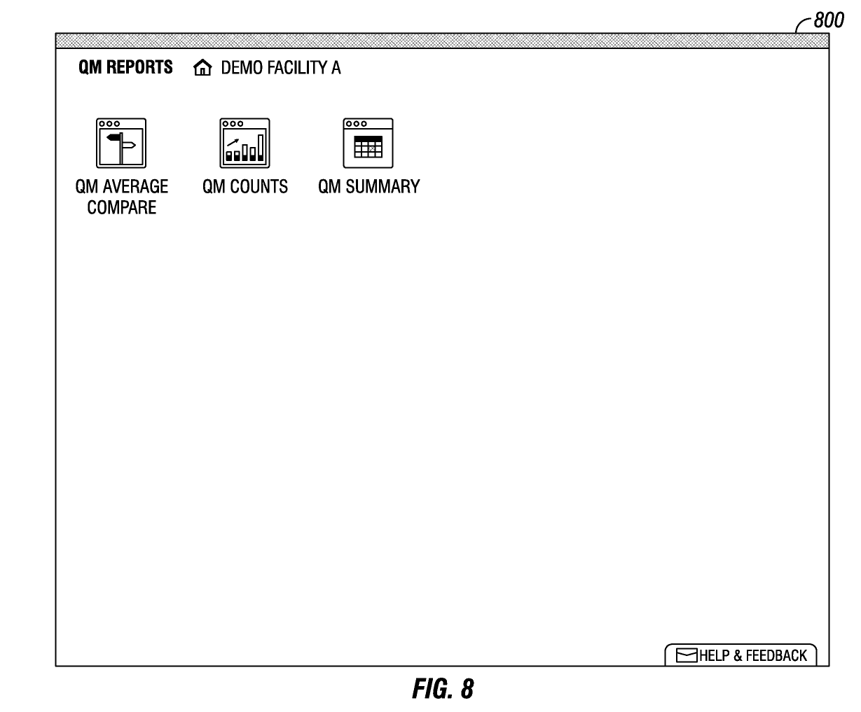

The slow, gradual deterioration of health conditions can go unnoticed by nurses and clinical supervisors in the skilled nursing setting, often resulting in medical crises that could have been prevented with early detection. In embodiments, the Live Quality Measures (e.g., reference the example Quality Measures Report 800 and the disclosure referencing FIG. 8) of the Decision Making Platform 100 may highlight clinical indicators of declining health conditions and bring them to the attention of clinical staff. This, coupled with specific intervention recommendations and a facility's real-time EMR data, may empower clinicians to provide timely, proactive care to keep residents healthy. With real-time, actionable clinical alerts, clinicians may adjust each resident's care accordingly, ensuring care is delivered to the right resident at the right time. This may also serve to improve billing accuracy and compliance. The Decision Making Platform 100, as described herein, may include a Clinical Dashboard 118 that provides clinicians with a single, detailed reporting interface that enables them to identify and intervene with at-risk residents, measure quality, and coordinate care. Unlike current systems providing skilled nursing analytics, the Decision Making Platform 100 does not require duplicate data entry because data may be pulled in real-time directly from an EMR 102 or other clinical data system. The Decision Making Platform 100 may also include a Financial Dashboard 120 that combines patient data from an EMR 102 with real-time scoring metrics, including but not limited to ADL data, to highlight opportunities for improved care, avoidance of reimbursement penalties, and/or increased reimbursements. The Clinical, Financial and all other Dashboards 118, 120, 122 described herein (collectively referred to as the “Dashboard” or “Dashboards” herein) may present analytic data and summaries for a single facility or across multiple facilities at a corporate level, and identify and display key trends in real-time to help facilities capitalize on reimbursement opportunities and reduce costly hospital readmissions.

In embodiments, the Decision Making Platform 100 may include a rules-based engine 116 capable of receiving EMR 102 or other data previously inaccessible to clinical and financial executives. This data may be analyzed for actionable insights that may have an impact on the return on investment (ROI) or other metrics of a healthcare facility.

By tailoring clinical alerts to a facility's specific policies and procedures, the Decision Making Platform 100 may provide clinical staff with meaningful alerts that will impact resident care. Clinical alerts derived from the Decision Making Platform 100 may be accompanied by specific intervention recommendations to help improve resident outcomes, making the 24-hour reporting process more comprehensive. In certain embodiments, a timing of the reporting process may be any selected timing, as the Decision Making Platform 100 enables configurable reporting, Dashboard updates, and/or alerting (collectively—Decision Making Platform 100 responses), including real-time Decision Making Platform 100 responses. In certain embodiments, 24-hour (daily) Decision Making Platform 100 responses are utilized, but responses may be additionally or alternatively performed, without limitation, at 72-hour intervals, at 8-hour intervals (e.g., a length of a shift, which may also be more or less than a 6-hour interval), monthly intervals, and/or 30-day intervals. In certain embodiments, responses may be at distinct intervals for distinct users of the system (e.g., a financial user versus a clinical staff member) and/or at selected times (e.g., in response to a reporting, fiscal, or regulatory deadline, before a shift begins, after a shift begins, before a holiday period, before a vacation period, etc.). In certain embodiments, responses may be at distinct intervals depending upon the type of response and/or the content of the response. For example, an alert may be performed on a different time cycle than a Dashboard update, and/or a response interval can be adjusted according to a timing component (e.g., fast decline of a resident condition versus a slow decline of a resident condition; a negative reporting outcome versus a positive reporting outcome, etc.) of the response.

In embodiments, using the Decision Making Platform 100, a director of nursing or other clinical department may manage care with real-time quality measures. The Decision Making Platform 100 may identify newly triggered quality measures for rapid clinical response. These insights may allow the facility to implement quality assessment and performance improvement (QAPI) measures to improve the care delivery model and to regain the regulatory ratings they may have lost. With the continual analytic capabilities of the Decision Making Platform 100 utilizing new data as it is generated, all shifts at work in a facility may be better able to deliver complete and consistent quality care by having access to the continuous quality monitoring, to communicate status across shifts, and/or to get a high quality overview of the facility status at the beginning of a shift, an ending of a shift, and/or at a selected interval during a shift.

In embodiments, the Decision Making Platform 100 may provide clinicians or others with customized resident alerts and suggested clinical interventions targeted to a given facility's patient population. This methodology may reduce the data “noise,” and improve the quality and accuracy of analytics relative to previously known and/or industry-standard alerts generated by EMRs. Customized alerts have been shown in studies to reduce hospital readmissions and the cost of care. The Decision Making Platform 100 may also provide customization of access to the Platform 100. For example, a service provider's access to patient data may be limited to only those patients for whom the service provider is authorized to provide services. In an example, a physical therapist visiting a long-term care facility to provide therapy may wish to access the Decision Making Platform 100 to check on the daily status of patients she will treat that day. The physical therapist may be associated with a payor, such as the private insurance company reimbursing her for physical therapy services. A payor code may be associated with the physical therapist and used to limit the therapist's access to patient data in the Decision Making Platform 100 to only those patients that are eligible for reimbursement by the payor. All other patient data may be blocked from access by the physical therapist.

In embodiments, data noise, error and inconsistencies may also be detected by the Decision Making Platform 100, resulting in data sets, analysis, and reports that are more accurate and less prone to propagate as errors (in reporting, processing, or treatment), or to cause delays in processing, such as during payor review or audit. Illogical data values and/or data value inconsistencies may be detected, filtered and corrected by the Decision Making Platform 100. For example, data relating to a patient's health state, such as the presence of a feeding tube, ostomy, or other condition may be used to check the quality and accuracy of other data fields in the aggregate patient data set 112. A patient with a feeding tube and/or ostomy should not have data values relating to normal eating and elimination, and the presence of data in such fields would be illogical and an error that should be corrected prior to analysis or reporting. Similarly, if a patient is coded in the aggregate patient data set 112 as being “self-supporting,” then it follows that elsewhere in the data set the same patient should not be coded as “impaired.” Upon detection of such illogical or inconsistent data, the Decision Making Platform 100 may send an alert and/or update a Dashboard with a notification, indicating review, correction, and/or reconciliation of the data is needed. The Decision Making Platform 100 may also place a hold on the data so that the inaccurate data is not propagated, for example through analysis, in a report, or in another Decision Making Platform 100 response, until the inaccuracy is corrected. Accordingly, certain negative outcomes may be avoided, such as sharing such data in a report and/or with a third party, such as a payor, which may lead to inaccurate payment, delayed processing of reimbursement, and/or improper treatment. Additionally or alternatively, auditing processes, whether internal or external to the treatment facility, are more likely to be working with valid data, and have a lower rate of error detection and subsequent negative outcomes.

The Decision Making Platform 100 may also allow standardization of data ranges and other codings that result from EMRs 102 or other data systems using data from disparate recording protocols and/or formats. For example, assisting a patient getting in or out of bed may be recorded using a 0, 1, or 2, where the number represents the number of caregivers needed to provide the assistance to the patient. One facility, or EMR 102, may record a sequential series of data entries throughout the day to represent each time assistance was needed. A second facility, or EMR 102, may instead sum the values recorded throughout a day and instead enter only a single value summarizing the assistance that a patient required. The Decision Making Platform 100 may detect such coding differences and standardize the values so that the data from these two patients may be evaluated in a similar manner, within a combined analysis, and the like. Additionally or alternatively, the Decision Making Platform 100 can configure the data such that output systems, such as payor systems, can receive data formatted or otherwise configured for their system. Standardization and/or accommodation may reduce computation and reporting time required to perform analyses using the data, and/or reduce data management and/or reimbursement issues with payors due to non-standard and/or disparately coded or formatted data.

In embodiments, the Decision Making Platform 100 may prioritize the most up-to-date and accurate EMR data 102 to facilitate clinicians predicting adverse health events and provide specific intervention recommendations to improve quality outcomes, without completing and/or submitting an MDS, and/or before completing and/or submitting an MDS.

In embodiments, the Decision Making Platform 100 may utilize data in an encrypted or unencrypted form. For example, data sent to the Platform 100 may be encrypted for security and/or data privacy. If the EMR 102 being used as a data source is hosted externally from the care facility that is using the Platform 100, access to that data may be similar to how the facility accesses it for other clinical purposes in the process of care (e.g., secure, encrypted, role-based).

In an embodiment, the Decision Making Platform 100 may be accessed via secure connections using HIPAA compliant security policies (e.g., username/password and/or login or access protocols), and daily intrusion detection reports may be utilized. Inactive accounts may be automatically deactivated to prevent unauthorized user access. A data center associated with the Decision Making Platform 100 may also have physical guards and multiple layers of on premise security and report annually on SOC-2 compliance, for example for HIPAA, PCI, PII, SPI, or other regulatory schemes or industry standards relevant to the data.

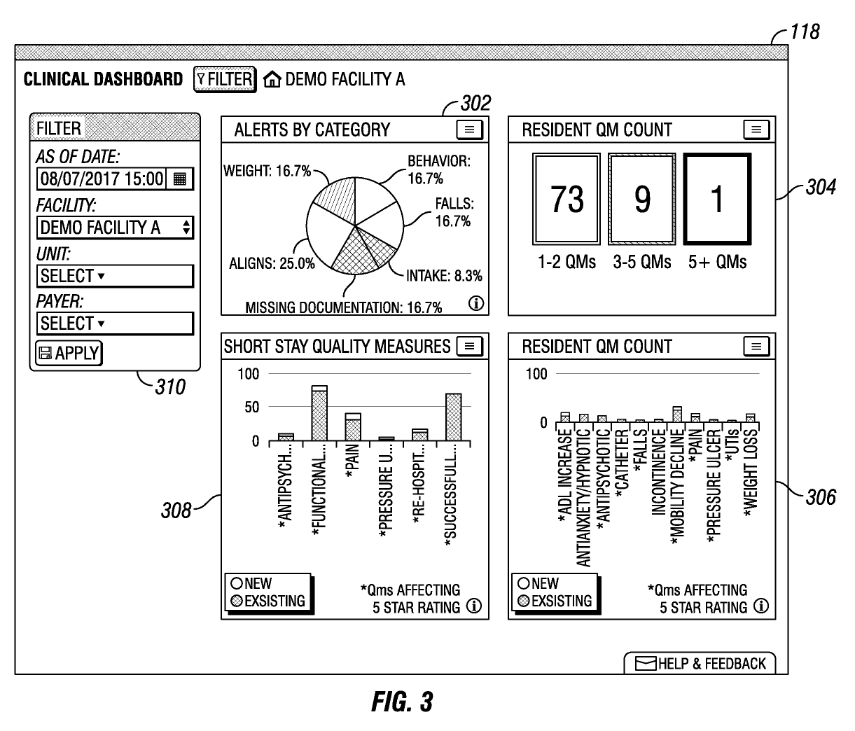

Referencing FIG. 3, an example Clinical Dashboard 118 provides clinical decision support for skilled nursing or other clinical fields. The example Clinical Dashboard 118 provides staff with detailed clinical alerts 302 and/or intervention recommendations (not shown) to ensure the appropriate care is administered, improving quality and outcomes of care, and reducing hospital readmissions. The example clinical alerts 302 include a summary of the number and/or type of alerts generated, but may additionally or alternatively provide a highlight (not shown) of specific alerts, allow for selection of certain patients and/or patient groupings, and/or allow for selection of certain facilities and/or locations within a facility, work shifts, and/or time periods. The Clinical Dashboard 118 may proactively manage quality data and outcomes with live quality measures, and use live clinical documentation to generate daily (or other time period) quality measure reports, descriptions, and/or visualizations. In the example of FIG. 3, the Clinical Dashboard 118 displays a quality measure resident summary 304 depicting resident counts grouped by a number of quality measures or issues active, and a quality measure histogram 306 depicting how many issues corresponding to particular quality measures are active. The presentation and organization of the quality measures is a non-limiting example, and in certain embodiments both the displayed quality measures, and the organization of those displays, is configurable by one implementing the Decision Making Platform 100 and/or by users. For example, the quality measure histogram 306 is depicted as an alphabetical listing, but a Pareto chart, severity order, or any other organization is possible. In certain embodiments, the Clinical Dashboard 118 includes a user configuration element, such as the user interface 310, to allow the user to filter, sort, summarize, and/or otherwise organize the Clinical Dashboard 118. In certain embodiments, a user may select a specific element (e.g., the quality measure resident summary 304) to configure and/or select options for that specific element.

Example and non-limiting quality measures include, without limitation, pain scores, usage of medications (including particular medications and/or types of medications), care indicia (e.g., bathing, bathroom usage, pressure ulcers, etc.), falls, infections (including particular infections or types of infections), weight gain or loss, particular types of treatment (e.g., catherization, colostomy, feeding tubes), immunizations, discharges and/or readmissions, improvement or decline in condition, and/or mobility factors. In certain embodiments, quality measures may be utilized in view of a patient condition—for example a patient in a coma may not have one or more quality measures calculated consistent with the condition of that patient. In certain embodiments, quality measures may be separated according to a patient group, category, or type, such as the separate short stay quality measure 308 depicted in the example of FIG. 3. These calculated care measures may allow staff the opportunity to place corrective measures in place to reduce these measures in real-time and/or reduce the reliance on outdated MDS data and CASPER reports. The Platform 100 may reduce or eliminate manual report pulls and duplicate data entry by accessing critical information in one centralized location with comprehensive, intuitive, web-based dashboard (e.g., the Clinical Dashboard 118).

In embodiments, the Decision Making Platform 100 may group alerts together by resident, by alert type, or some other grouping criterion, to allow nurses to access all of a resident's alerts in one place. By simplifying the reports and alerts, nursing staff may better understand each resident's clinical scenario to deliver more efficient and effective care. Under current methods and systems, facilities are often dependent upon MDS completion to view quality measure results. However, the Dashboards 118, 120, 122 of the Decision Making Platform 100, as described herein, may display live quality measures to help facilities proactively manage their quality data and outcomes, using the live clinical documentation to generate measures at any selected time period, such as daily or even real-time. These calculated care measures may allow staff the opportunity to place corrective measures in place to improve these measures in real-time. Facilities may be able to use the Decision Making Platform 100 to identify which residents recently triggered a given measure or have been in a measure, for example, over the last seven days. These insights may allow the facility to implement quality assurance and performance improvement (QAPI) measures to improve the care delivery model, and/or to maintain or regain a rating and/or reward level.

In embodiments, the analytic output of the Decision Making Platform 100 may be automatically sent to other computing systems associated with a facility, including but not limited to billing systems, inventory systems, electronic health records (EHR) systems, pharmacy systems, laboratory systems, scheduling system, charting system, incident reporting system, and/or bed management system. In an example, the Decision Making Platform 100 may receive data indicating that a patient or plurality of patients have had worsening health conditions related to eating and elimination as indicated, for example, by ADL, quality measures, or other scores. This information may be reported by the Decision Making Platform 100 as an alert to a plurality of other computing systems associated with the facility. For example, the Decision Making Platform 100 may send an alert to a scheduling system indicating the time at which a caregiver should be reminded to test and enter fluid input and output for a patient. Such a reminder may instead, or also, be sent to a remote client device, such as a smart phone, that is associated with the caregiver. The alert may be transmitted over a communication channel to the remote client device associated with the caregiver based upon a destination address and associated with the remote client device, wherein the alert activates the user interface of the remote client device, causing the alert to display on the remote client device and to enable connection with the Decision Making Platform 100 when the remote client device is activated. Continuing the example, a laboratory system may also receive an alert from the Decision Making Platform 100 indicating that a patient is to have fluid input and output measured at a given time, and note the expected arrival of a fluid specimen (e.g., a urine sample) to arrive at a time proximate to the measurement of the fluid input and output. A failure of the specimen to arrive may prompt an additional alert to be sent to the caregiver, such as a request to confirm that the fluid measurement was taken. Still continuing the example, the Decision Making Platform 100 may also send an alert to a billing system that is associated with the facility, indicating that the health state for the patient is in flux and trending downwards (a potential indicator of a change in the patient's reimbursement status), and indicating in the system to revisit the reimbursement coding or other information following the updated entry of the patient's clinical data into the Decision Making Platform 100. Any alerts in the system, including without limitation any one or more of the foregoing example alerts, may additionally or alternatively be provided on a Dashboard 118, 120, 122, for example any Dashboard 118, 120, 122 that the alert target (e.g., a caregiver, laboratory system administrator, billing system administrator, etc.) has access to, is periodically checked by the alert target, and/or that is configured to provide information relevant for the alert target.

In embodiments, intervention recommendations may be delivered directly to a facility's care team in a single report, in a number of reports, as an alert, and/or provided on a Dashboard 118, 120, 122. The reporting or other notification of intervention recommendations may be segmented by, for example, the units of a facility and distributed to the appropriate staff so they can make their rounds and have an immediate positive impact on resident care. While EMRs produce a limited set of generic clinical alerts, an example Platform 100 provides analytics-based alerts may be tailored to fit a facility's unique policies and procedures. For example, when a resident diagnosed with congestive heart failure has newly documented edema and weight gain, the Platform 100 may setup a customized alert or other notification to clinicians when the event occurs, ensuring the appropriate care is provided to improve patient outcomes, and/or avoid readmission or re-hospitalization.

In embodiments, the Dashboards 118, 120, 122 enable decision makers to choose an assessment reference date (ARD) that's best for each resident. In conjunction with therapy, decision makers may take greater control of the MDS process and make informed, meaningful decisions using the Dashboards 118, 120, 122 to show, for example, MDS coordinators real-time resource utilization groups (RUGs, e.g., RUG-III, RUG-IV, etc.) and/or ADL score distributions to help in setting optimal ARDs to plan for updates to staffing, checks on patient care or quality measures, and/or optimize reimbursement. For example, previously known systems for skilled nursing facilities wait until the end of the month to see which residents were billed at A, B, and C ADL levels. With the real-time ADL coordination of the Decision Making Platform 100, a user may track each resident's ADL score in real-time before the MDS is created so they may identify which residents may have been miscoded before the next ARD, to plan for aggregate care needs of the patient population in real-time, and/or to plan patient care, treatment, and status check timing.

In embodiments, the Decision Making Platform 100 may identify opportunities for better, more accurate documentation and help MDS coordinators focus on appropriate ARD setting practices. The Decision Making Platform 100 may also identify residents with declining functional levels based on real-time ADL scores, helping clinicians to focus on specific resident needs to reduce the risk of costly hospital readmissions or further declines in functional levels. For example, a daily rehab “huddle” process may be enhanced and simplified with daily RUG-IV ADL scoring. Setting ARDs collaboratively may improve accuracy, patient care quality, and reimbursements levels at skilled nursing or other healthcare facilities.

As depicted in FIG. 3, the Clinical Dashboard 118 of the Decision Making Platform 100 may present a user a series of menus on which the underlying data (e.g., data from EMRs 102) may be filtered or otherwise presented. Filtering criteria may include, but is not limited to, date, facility, unit within a facility, payer, or some other criterion. Filtered data may be presented in the Dashboard 118 as “cards,” or separate data summary fields (e.g., 302, 304, 306, 308). In certain embodiments, the use of filtering, sorting, and/or other manipulation of data summary fields provides for more efficient computing operations (e.g., limiting a data set to data of interest for the user, thereby reducing computing resources for communicating and/or storing data, and for processing and analyzing the data). In certain embodiments, the use of filtering, sorting, and/or other manipulation of data summary fields provides for greater utility of the Dashboard 118 to the user, improving response times of the user to the data, and improving patient care outcomes. A user may use the Dashboard 118 to set the types of alerts to send and/or display on the Dashboard 118.

Alerts may be categorized and selected by alert category. Alert categories may include, but are not limited to, clinical categories, such as weight or behavioral variables of a resident, incidents, such as falls, procedural indicators, such as resident intake, or some other category. Alert summaries displayed within a card in the Dashboard 118, 120, 122 may be sorted by category and/or include only alerts of a certain category. In an example, the “alerts by category” card 302 may focus on the last 24 hours (or other selected time period and/or response period) of clinical alerts that triggered on the current resident population in a facility. The clinical alerts may be created by the live documentation from an EMR(s) and the values that support the alert may be noted in each column of the display. Suggested interventions or clinical pathways may be displayed that clinicians should consider to reduce the effects of, or to respond appropriately to, the clinical alert. In certain embodiments, without limitation, an alert may be financial (e.g., an incident or change in the data affecting the financial position, projection, or a financial target), related to transitions of care (e.g., discharge, admission, readmission, etc.), and/or administrative (e.g., a staffing change indicated in the data, a follow-up of any other alert due to an unresponsive or out-of-office recipient, an erroneous record or other aspect of the data where it may be desirable to address the issue).

In embodiments the Dashboard 118, 120, 122 may present quality measures, including but not limited to measures of patent function, pain, vital signs, re-hospitalization events, or some other criterion. These may be further categorized by, for example, new patients and existing patients, long and short stay patients, or some other criterion. Cumulative summaries and comparisons may be provided, such as quality measure summaries, counts and frequencies. In an example, a card 308 summarizing short stay data may include displays of Short Stay Quality Measures, such as percentages that have triggered based on actual clinical documentation, which will be more responsive than reliance on the last submitted MDS. An example Dashboard 118 includes one or more Quality Measures triggered or determined in accordance with CMS MDS 3.0 definitions and criteria, and/or in accordance with any other applicable regulatory scheme, industry standard, and/or applicable policy. An example includes indicators flagged if a quality measure that affects a rating or other regulatory or contractual measure. An example includes a list of residents may be separated into groups, such as: New Patient and Existing Patient. New Patient triggers may be provided when a resident enters the quality measure within, for example, the last 7 days. Existing Patient triggers may be provided when a resident continues to trigger the quality measure after 7 days. A resident may fall out of the quality measure, for example, after 14 days of non-triggering documentation in the clinical record. The number of days since last MDS may help prioritize which residents are at risk for triggering the quality measure, for example on a CASPER Report.

In embodiments, the Dashboard 118, 120, 122 may facilitate a number of reporting and exporting features, including but not limited to exporting a spreadsheet or other delimited-type data set, exporting to .pdf or other document type, or some other reporting type.

In one example, in the United States, the Improving Medicare Post-Acute Care Transformation (IMPACT) Act will require skilled nursing providers to report standardized resident assessment data and quality measures. With the IMPACT Act, quality measures will have a greater influence on Medicare reimbursements as skilled nursing providers will be required to report on more quality measures. As this reform to the skilled nursing payment model increases the importance of quality of care to the reimbursement process, facilities must improve the quality of care provided and increase the accuracy of their documentation. An example Decision Making Platform 100, as described herein, may help skilled nursing and other healthcare providers focus on residents' needs and quality of care, which is the primary goal of the IMPACT Act. Any other quality initiative, program, and/or regulation can be similarly supported by embodiments of the Decision Making Platform 100.

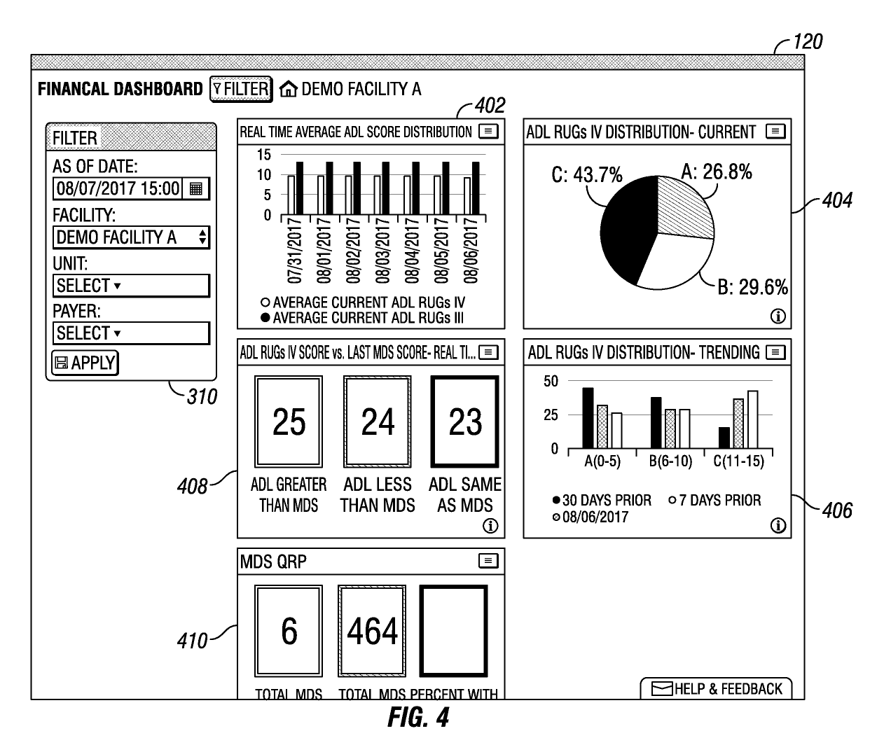

Referencing FIG. 4, in embodiments the Financial Dashboard 120 of the Decision Making Platform 100 may display resident care data determined from an EMR 102 and display real-time ADL scoring metrics (e.g., card 402), highlighting opportunities for increased reimbursements. A user may be able to view high-level ADL distributions at both a facility, on site, and at the corporate level (e.g., at a centralized office) over a selected period, for example, a 30-day window, using the Financial Dashboard 120. Key trends may be identified and opportunities for optimizing reimbursements discovered.

In embodiments, for example during the implementation of the Decision Making Platform 100 and/or at selected times, the Decision Making Platform 100 may perform a baseline assessment of, for example, the past 12 months of documentation and ADL scores. Accurate documentation of ADLs is a significant component of reimbursement. For example, the ADL level is what determines the reimbursement rate paid to a facility by Medicare and Medicaid. For example, the difference between an A-level ADL score and a B-, or C-level score can be up to $95 per resident per day. Accordingly, the financial impact of accurate ADL calculations is significant. For long-term care facilities to receive the appropriate reimbursement amount, it's critical that resident care is documented correctly. Due to a lack of knowledge and understanding of the coding structure, clinicians often under code residents, landing them in an artificial, higher-functioning ADL level and causing the facility to miss out on reimbursement funds for the care provided. Similarly, a lack of accuracy of the ADL level on the low end can result in penalties or other negative consequences. The Decision Making Platform 100, as described herein, may receive pertinent resident information from any EMR 102 or other system, and provide a summary of a long-term care facility's financial standing in the Financial Dashboard 120. The Financial Dashboard 120 may also track high-level ADL distributions at a facility and/or corporate level over, for example, a 30-day window, so you can easily identify key trends.

The Financial Dashboard 120 of the Decision Making Platform 100 may present a user a series of menus on which the underlying data (e.g., data from EMRs 102) may be filtered, sorted, summarized, and/or otherwise organized. Filtering criteria may include, but is not limited to, date, facility, unit within a facility, payer, or some other criterion. The cards 402, 404, 406, 408, 410 of the Financial Dashboard 120 may include, but are not limited to, Average ADL Score Distribution, RUGs IV Score, RUGs IV Score versus last MDS Score, MDS QRP, ADL RUGs IV Distribution, ADL RUGs IV Trend, or some other data or analytic summary.

In an example, an Average ADL Score Distribution 402 may calculate the LIVE RUGs IV and III ADL Scores on every resident everyday at a selected time (e.g., midnight). This score may incorporate the 4 Late Loss ADLs (Bed Mobility, Transfers, Eating, and Toileting) from a selected time period (e.g., the last 7 days) of the point-of-care documentation. Each episode of care may be captured and calculated using the current MDS 3.0 RAI or other applicable methodologies. In the example, a drill down of the last 7 days of documentation may be viewed by, for example, clicking on the resident, and the name of the staff that recorded that event can be viewed, for example, by hovering over the entry. The example interfaces are non-limiting, and any data drill-downs, filtering criteria, sorting options, or other data manipulation and organizational features are contemplated herein. The example of FIG. 4 includes a user interface 310 to support certain functions of filtering, sorting, and/or summarizing the information on the Financial Dashboard 120.

In an example, a card 408 summarizing RUGs IV Score versus last MDS Score may compare the Live RUGs IV and III ADL scores to the most recent MDS submitted, and assist in identifying which residents are declining, improving, or staying the same since last assessed. In an example, each category may be viewed separately by clicking on the description, and a list of residents can be viewed. The last MDS 4 Late Loss ADLs (Bed Mobility, Transfers, Eating and Toileting) values may be exposed and compared, for example by clicking on a resident's name.

In an example, a card 410 summarizing MDS QRP may calculate the number of qualified QRP MDSs submitted that contain inappropriate dashes in accordance with the Quality Reporting Program Measures. An example includes a threshold of acceptance set at <20% without receiving a 2% APU reduction. The actual thresholds utilized may depend upon applicable regulations, contractual obligations, internal policies and goals, or the like. In certain embodiments, a card 410 may be configured for selectable thresholds. The MDSs that contain dashes or other non-compliant aspects may be viewed by clicking on the card 410 for further review.

In an example, a card 404 summarizing ADL RUGs IV Distribution may segment the resident population into RUGs IV ADL End-split associations. The End-splits may be A (ADL score 0-5), B (ADL Score 6-10) and C (ADL Score 11-16). These end-splits may be created based on the current ADL Score of the resident, for example from the last 7 days of point-of-care data using the MDS 3.0 RAI or other methodologies. Low RUGs IV ADL scores denote a greater independence of the resident with the 4 Late Loss ADLs (Bed mobility, Transfers, Eating and Toileting), and the higher the score the greater the dependence for those ADLs. A drill down into each category may be provided to review those resident groupings.

In an example, a card 406 summarizing ADL RUGs IV Trend may depict the trends in the RUGs IV ADL end-split distribution, for example from the present to 7 days and/or 30 days ago. These end-splits may be created based on the current ADL Score of the resident from the last 7 days of point-of-care date using the MDS 3.0 RAI or other methodologies. The acuity of the resident population may be viewed and monitored in this card 406.

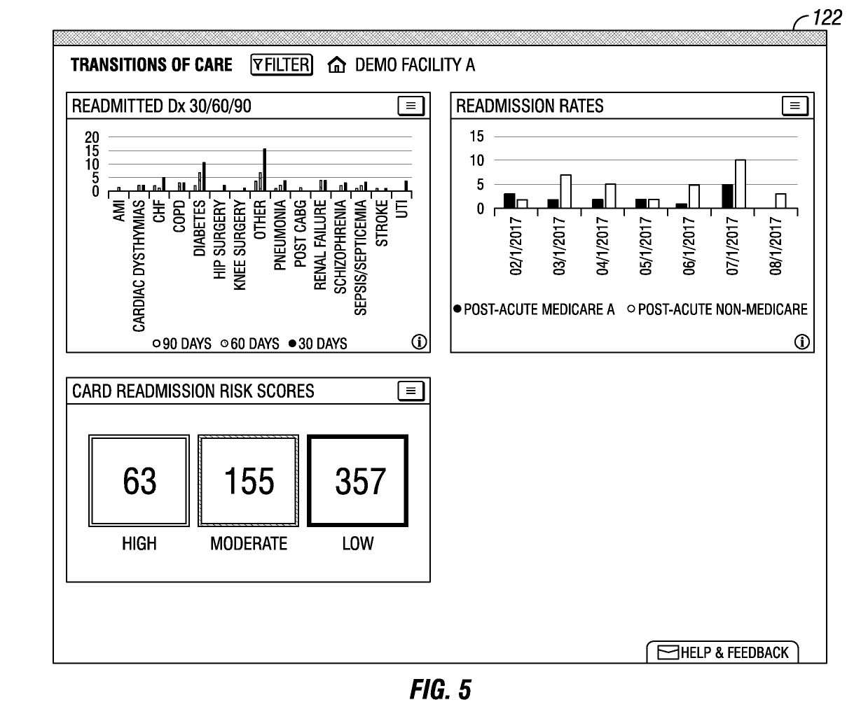

Referencing FIG. 5, an example Decision Making Platform 100 may include a Transitions of Care Dashboard 122 that includes cards 502, 504, 506 presenting and/or displaying data relating to Readmissions, Readmission Risk Scores, and/or Readmission Rates. In certain embodiments, information presented on the Transitions of Care Dashboard 122 includes any readmission response value, including one or more of a treatment recommendation value (e.g., a treatment and/or treatment timing determined to reduce readmission risk), an intervention recommendation value (e.g., a treatment change or option, and/or care change or option determined to reduce readmission risk), and/or a readmission risk report (e.g., a report, visualization, and/or card displaying data related to the readmission risk of a resident, a group of residents, a facility, etc.). In an example, a card summarizing Readmission 502 may display the active diagnoses/comorbidities that one or more readmission patients have (or previously had) upon discharge. This may allow a user and/or an analytic engine 114 to identify possible leading causes for the readmission. If a discharged resident has multiple diagnoses on the graph, the resident may be placed under each diagnosis to help further determine a root cause.

In an example, a card 506 summarizing Readmission Risk Scores may produce a weighted score, for example based on clinical alerts of the last 72 hours, length of stay, acuity scoring, active diagnoses and comorbidities, or some other criterion to risk score the likelihood of a readmission.

In an example, a card 506 summarizing Readmission Rates may display Live 30-Day (or other selected period) Readmission Rates. This rate may be calculated by identifying individual residents admitted to a facility after an inpatient hospital stay during a given period, for example calculated monthly, and following them for 30 days. The report may depict trends in Post-Acute Medicare A, Post-Acute Non-Medicare A and All Resident Readmissions in the current month. Each readmission may be viewed by selecting the desired month and type. An example Transitions Of Care Dashboard 122 is configured to allow interaction with the card 506, for example allowing a drilldown for each rate category, and for each resident (e.g., by clicking the resident name or other feature of the interface). In certain embodiments, each resident can be displayed to show the real-time clinical alerts that occurred within a selected time period (e.g., 72 hours) prior to discharge.

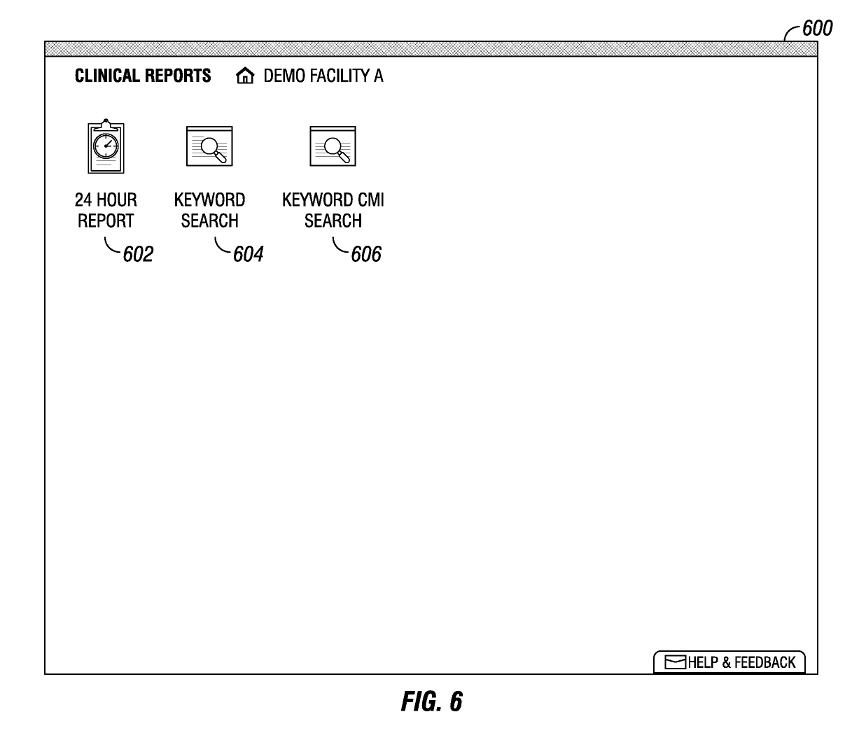

Referencing FIG. 6, an example Decision Making Platform 100 may provide a clinical reporting tool 600. An example clinical reporting tool 600 provides reports for a selected period (e.g., 24 hours in the example of FIG. 6), relating to a facility's operation 602, case mix index (CMI) searching 604, and keyword searching 606. The selected reports from the clinical reporting tool 600 are non-limiting examples, and reports can be configured for any purpose. In certain embodiments, the clinical reporting tool 600 is accessible from a Dashboard 118, 120, 122, and/or can be utilized to access a Dashboard 118, 120, 122.

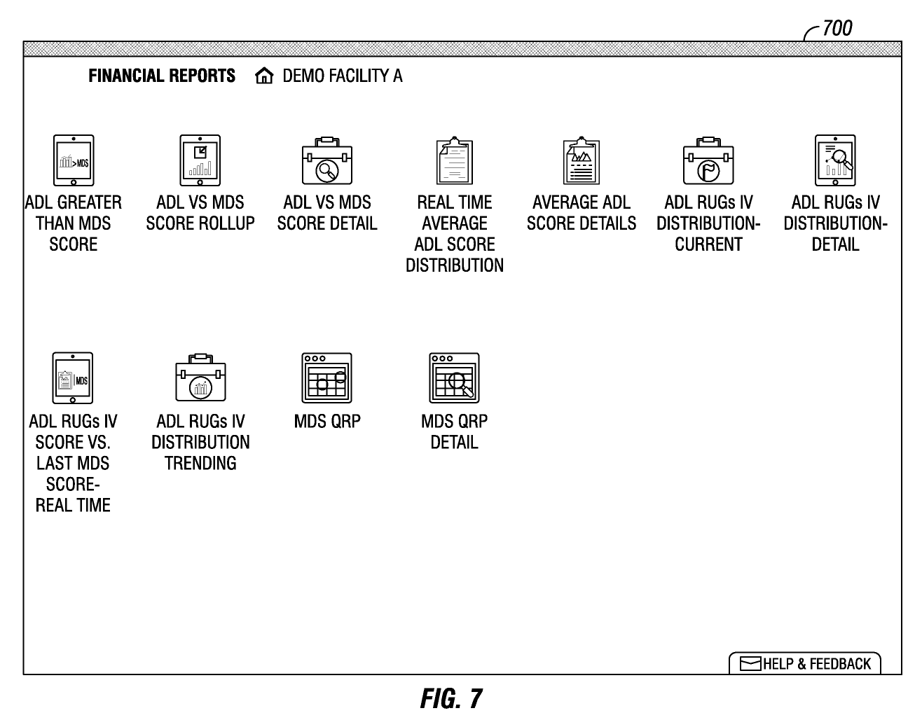

Referencing FIG. 7, an example Decision Making Platform 100 may provide a financial reporting tool 700. The example financial reporting tool 700 provides reports for selected time periods and various financial aspects relating to a facility, a group of facilities, or the like. The example report options depicted on FIG. 7 are non-limiting and illustrative. In certain embodiments, the financial reporting tool 700 is accessible from a Dashboard 118, 120, 122, and/or can be utilized to access a Dashboard 118, 120, 122.

Referencing FIG. 8, an example Decision Making Platform 100 may provide a quality measures reporting tool 800. The example quality measures reporting tool 800 provides reports for selected time periods and various quality measures relating to a facility, a group of facilities, or the like. The example report options depicted on FIG. 8 are non-limiting and illustrative. In certain embodiments, the quality measures reporting tool 800 is accessible from a Dashboard 118, 120, 122, and/or can be utilized to access a Dashboard 118, 120, 122.

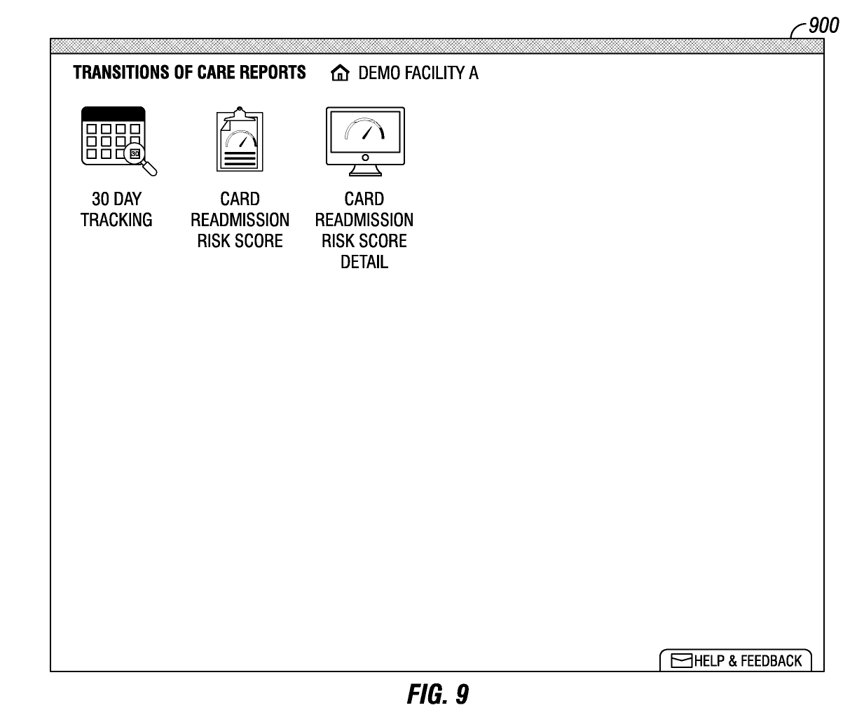

Referencing FIG. 9, an example Decision Making Platform 100 may provide a transitions of care reporting tool 900. The example transitions of care reporting tool 900 provides reports for selected time periods and various indicators of readmission risk and readmission risk management relating to a facility, a group of facilities, or the like. The example report options depicted on FIG. 9 are non-limiting and illustrative. In certain embodiments, the transitions of care reporting tool 900 is accessible from a Dashboard 118, 120, 122, and/or can be utilized to access a Dashboard 118, 120, 122.

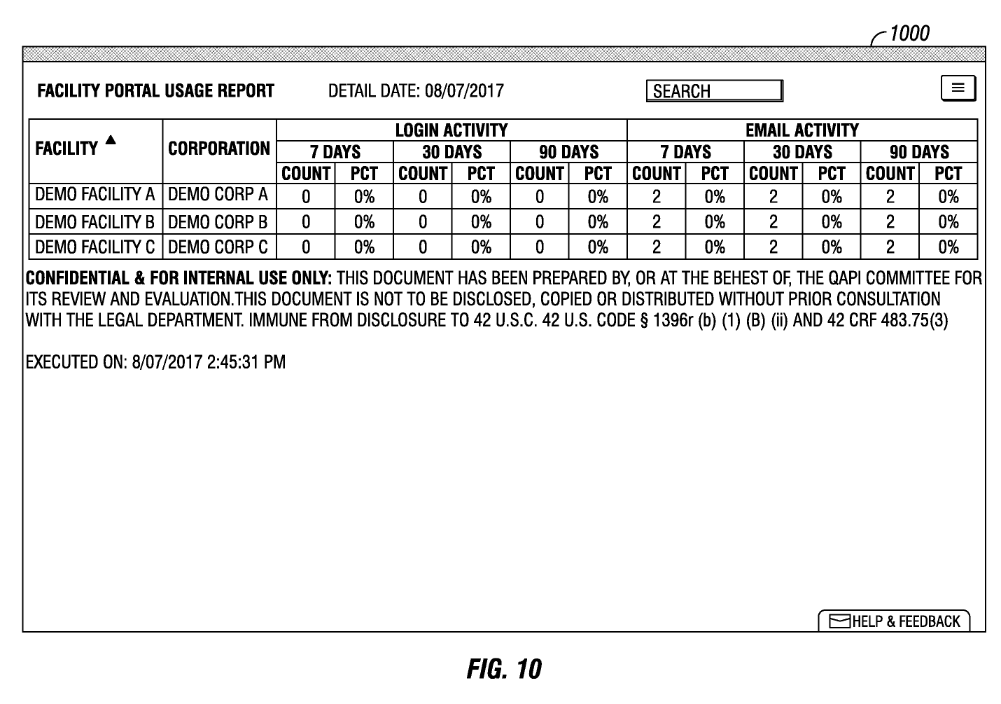

Referencing FIG. 10, an example Decision Making Platform 100 may provide a facility portal usage reporting tool 1000, for example to report on various measures of a portal usage interacting with a Decision Making Platform 100. The example facility portal usage reporting tool 1000 provides reports for selected time periods and various portal usage activities relating to a facility, a group of facilities, or the like. The example report options depicted on FIG. 10 are non-limiting and illustrative. In certain embodiments, the facility portal usage reporting tool 1000 is accessible from a Dashboard 118, 120, 122, and/or can be utilized to access a Dashboard 118, 120, 122.

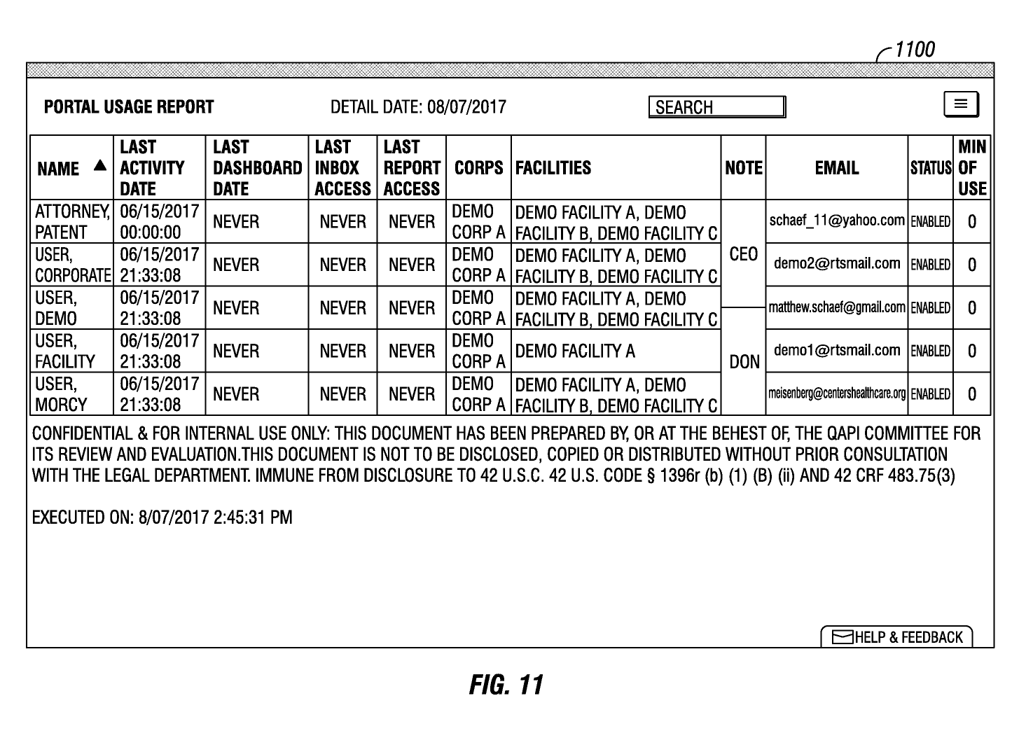

Referencing FIG. 11, an example Decision Making Platform 100 may provide a portal usage reporting tool 1100. The example portal usage reporting tool 1100 provides reports for selected time periods and various quality measures relating interactions with the Decision Making Platform 100, such as administrative, super-user, and/or any interactions including facility users and/or client users. The example report options depicted on FIG. 11 are non-limiting and illustrative. In certain embodiments, the portal usage reporting tool 1100 is accessible from a Dashboard 118, 120, 122, and/or can be utilized to access a Dashboard 118, 120, 122.

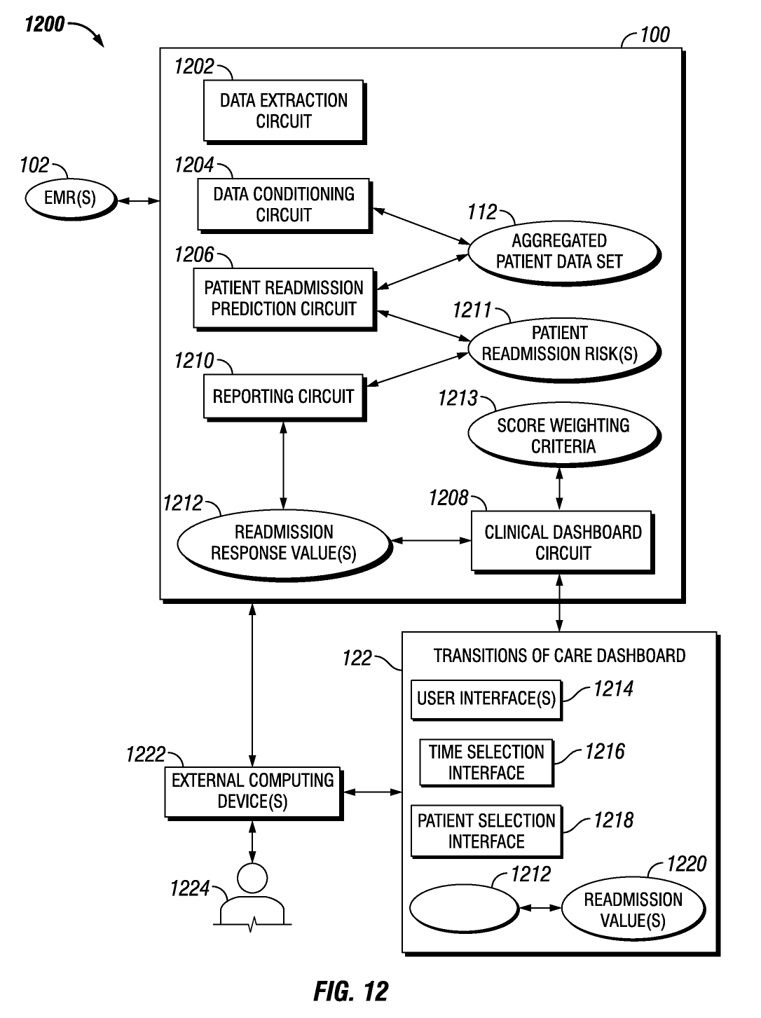

Referencing FIG. 12, an example system 1200 includes a decision making platform 100 having a number of circuits structured to functionally execute certain operations of the system 1200. The example system 1200 includes a data extraction circuit 1202 that interprets a number of EMRs 102, and a data conditioning circuit 1204 that determines an aggregated patient data set 112 in response to the number of EMRs 102. In certain embodiments, the data conditioning circuit 1204 performs standardization and/or accommodation of data, for example where distinct sources of EMRs 102 have disparate formatting and/or recording protocols, and/or where distinct clients of the decision making platform 100 require disparate formatting and/or recording protocols of data and/or reports provided by the decision making platform 100.

The example system 1200 further includes a patient readmission prediction circuit 1206 that determines a readmission risk 1211 for a patient in response to the aggregated patient data set 112, for example utilizing any of the operations or procedures to determine and/or estimate patient readmission risks described throughout the present disclosure. The example system 1200 further includes a reporting circuit 1210 that provides a readmission response value 1212 in response to the readmission risk 1211 for the patient. A readmission response value 1212 includes, without limitation, any risk description for readmission of a patient, and/or a recommendation of a treatment and/or an intervention (e.g., changing the timing and/or planned activity of a treatment or care for the patient) for the patient. Further non-limiting examples of a readmission response value 1212 include: a treatment recommendation value, a patient alert value (e.g., one or more alerts relating to the patient), a treatment time value (e.g., a scheduled time, a changed time, a time window, etc.), an intervention recommendation value, and/or a readmission risk report corresponding to the patient (and/or including additional patients, and/or an aggregated risk report for a group of patients, and/or patients for a facility or group of facilities). In certain embodiments, the reporting circuit 1210 provides the readmission response value 1212 to a user 1224 (e.g., via external computing device 1222), and/or to any portion of the system 1200, a client, a payor, and/or a government entity. In certain embodiments, the reporting circuit 1210 provides a report including the readmission response value 1212 any portion of the system 1200, a client, a payor, and/or a government entity.

The example system 1200 further includes a clinical dashboard circuit 1208 that implements a transitions of care dashboard 122. A transitions of care dashboard 122 may be any dashboard as described throughout the present disclosure. The example transitions of care dashboard 122 depicts the readmission response value(s) 1212 at least partially available thereon for access by the user 1224, and/or one or more readmission values 1220, which may be displayed as a card on the transitions of care dashboard 122, and/or may be the readmission response value(s) 1212 and/or values determined from the readmission response value(s) 1212. In certain embodiments, the readmission rates include at least one of: a 30-day readmission rate value, a monthly readmission rate value, a readmission rate value for a selected time period, and/or a number of readmission rates each corresponding to a selected patient grouping category (e.g., having a certain diagnosis, a certain ADL classification, a certain RGU classification, a certain quality measure indicated, and/or located at one or more selected facilities). In certain embodiments, the transitions of care dashboard 122 includes a user interface 1214 accessible to at least one external computing device 1222, and further includes at least one of: a number of readmission scores corresponding to a number of readmission events, readmission rates 1220 corresponding to at least a portion of the aggregated patient data set 112, a number of diagnosis values (e.g., diagnosed conditions, quality measures, and/or alert values) corresponding to a number of readmission events, and/or a number of comorbidity values corresponding to a number of readmission events.

In certain embodiments, the transitions of care dashboard 122 further includes a time selection interface 1216, for example allowing the decision making platform 100 and/or the user 1224 to define time values of interest for the data on the transitions of care dashboard 122. In certain embodiments, the clinical dashboard circuit 1208 filters, groups, and/or sorts data on the transitions of care dashboard 122 in response to the selection of the time selection interface 1216.

In certain embodiments, the transitions of care dashboard 122 further includes a patient selection interface 1218, for example allowing the decision making platform 100 and/or the user 1224 to define patient values (e.g., a patient name and/or number) or patient grouping categories. In certain embodiments, the clinical dashboard circuit 1208 displays the readmission response value 1212 and/or the readmission values 1220 in response to a patient selection of the patient selection interface 1218. In certain further embodiments, the readmission values 1220 include at least one of: active diagnoses corresponding to the patient selection, comorbidities corresponding to the patient selection, a readmission rate value corresponding to the patient selection, a readmission score value corresponding to the patient selection, and/or a patient alert value history corresponding to the patient selection.

In certain embodiments, the readmission values 1220 are weighted values, such as weighted scores, and/or averaged values with weighting applied. In certain embodiments, the readmission values 1220 include a number of readmission scores corresponding to a patient selection, wherein the readmission scores are weighted scores. In certain embodiments, the weighted readmission scores and/or values are weighted according to a score weighting criteria 1213, which may be provided by the clinical dashboard circuit 1208 and/or a user 1224. Example an non-limiting score weighting criteria 1213 include at least one of: a number of patient alert values, a number of patient alert values corresponding to a predetermined time period, a length of stay description for the aggregated patient data set (e.g., short stays vs. long-term patients), a set of diagnoses corresponding to a portion of the aggregated patient data set 112, a set of comorbidities corresponding to a portion of the aggregated patient data set 112, and/or a time dependent value (e.g., the prior 24 hours, 72 hours, 1 week, 1 month, a time-based rate of change, etc.) for any of these.

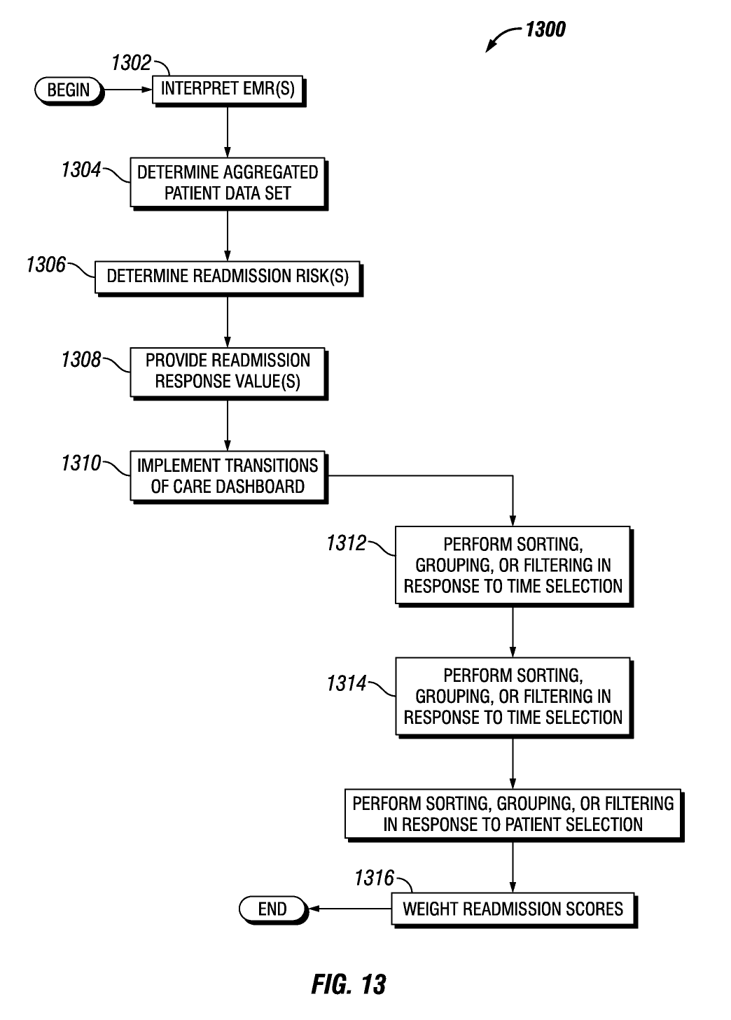

Referencing FIG. 13, a schematic flow diagram depicts an example procedure 1300 for providing a readmission response value. The example procedure 1300 includes an operation 1302 interpret a number of electronic medical records (EMRs), an operation 1304 to determine an aggregated patient data set in response to the number of EMRs, an operation 1306 to determine a readmission risk for a patient in response to the aggregate patient data set, and an operation 1308 to provide a readmission response value in response to the readmission risk for the patient. In certain embodiments, the example procedure 1300 includes an operation 1310 to implement a transitions of care dashboard, where the transitions of care dashboard includes a user interface accessible to at least one external computing device, and where the transitions of care dashboard further includes: a number of readmission scores corresponding to the number of readmission events, readmission rates corresponding to at least a portion of the aggregated patient data set, a number of diagnosis values corresponding to the number of readmission events, and/or a number of comorbidity values corresponding to the number of readmission events. An example procedure 1300 further includes the operation 1310 to implement a transitions of care dashboard.

An example transitions of care dashboard includes a user interface accessible to at least one external computing device, and readmission rates corresponding to at least a portion of the aggregated patient data set. The example readmission rates include one or more of: a 30-day readmission rate value, a monthly readmission rate value, a readmission rate value for a selected time period, and/or a number of readmission rates each corresponding to a selected patient grouping category. An example transitions of care dashboard further includes a time selection interface, where the example procedure 1300 further includes an operation 1312 to perform sorting, grouping, and/or filtering the transitions of care dashboard in response to a selection of the time selection interface. An example transitions of care dashboard further includes a patient selection interface, where the example procedure 1300 further includes an operation 1314 to display a number of readmission values in response to a patient selection of the patient selection interface. In certain embodiments, readmission values include one or more of: active diagnoses corresponding to the patient selection, comorbidities corresponding to the patient selection, readmission rate values corresponding to the patient selection, a readmission score value corresponding to the patient selection, and/or a patient alert value history corresponding to the patient selection.

An example procedure 1300 includes the operation 1310 to implement a transitions of care dashboard, where the transitions of care dashboard further includes a user interface accessible to at least one external computing device, and a number of readmission scores corresponding to the number of readmission events, where the number of readmission scores include weighted scores. An example procedure 1300 further includes an operation 1316 to weight the number of readmission scores according to one or more of the following criteria: a number of patient alert values, a number of patient alert values corresponding to a predetermined time period, a length of stay description for the aggregated patient data set, a set of diagnoses corresponding to a portion of the aggregated patient data set, a set of comorbidities corresponding to a portion of the aggregated patient data set, and/or a time dependent value for any of the foregoing.

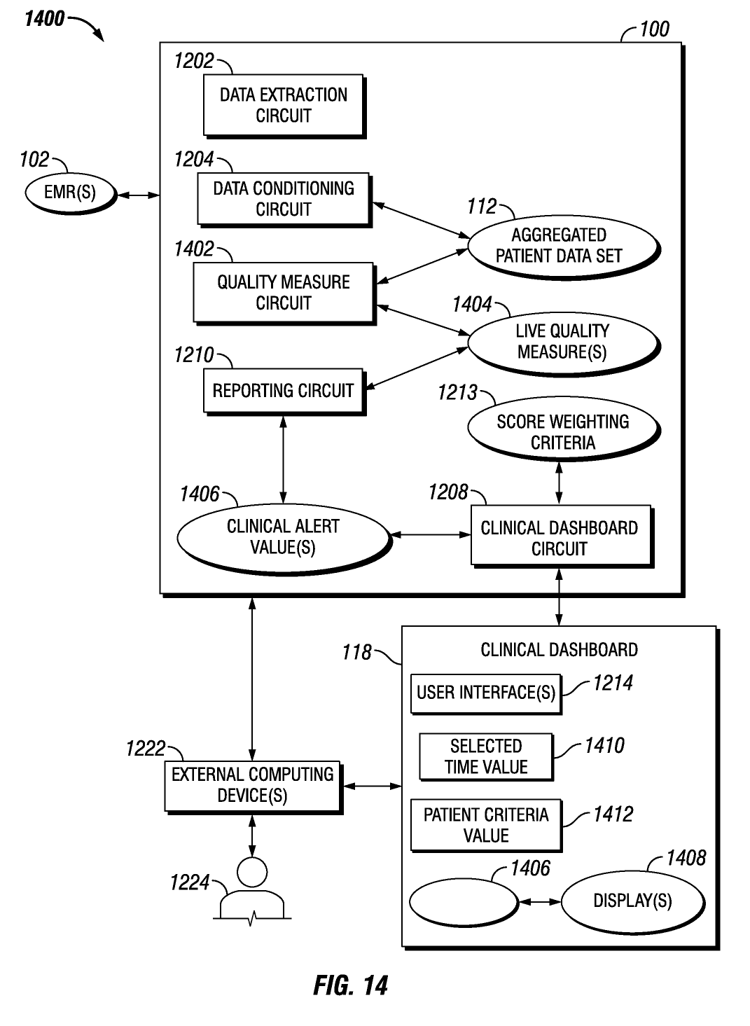

Referencing FIG. 14, an example system 1400 includes a decision making platform 100 having a number of circuits structured to functionally execute certain operations of the system 1400. The example system 1400 includes a data extraction circuit 1202 that interprets a number of EMRs 102, and a data conditioning circuit 1204 that determines an aggregated patient data set 112 in response to the number of EMRs 102. In certain embodiments, the data conditioning circuit 1204 performs standardization and/or accommodation of data, for example where distinct sources of EMRs 102 have disparate formatting and/or recording protocols, and/or where distinct clients of the decision making platform 100 require disparate formatting and/or recording protocols of data and/or reports provided by the decision making platform 100.

The example system 1400 further includes a quality measure circuit 1402 that determines a live quality measure 1404 corresponding to a patient in response to the aggregated patient data set 112, for example utilizing any of the operations or procedures to determine and/or estimate quality measures described throughout the present disclosure. The example system 1200 further includes a reporting circuit 1210 that provides a clinical alert value 1406 in response to the live quality measure 1404 for the patient. Example and non-limiting live quality measures include any clinical indicator of declining health for the patient, and/or any other quality measures described throughout the present disclosure. An example data conditioning circuit 1204 further filters the aggregated patient data set 112, and the reporting circuit 1210 provides the clinical alert value 1406 further in response to the filtered aggregated patient data set 112. Example and non-limiting filters of the data conditioning circuit 1204 include one or more of a patient grouping filter, a facility filter, and/or a time-based filter. For example, where the clinical alert value 1406 is to be provided to a particular person, an example data conditioning circuit 1204 filters the aggregated patient data set 112 to include only patients relevant to that particular person. An example data conditioning circuit further corrects at least one of the EMRs 102 before determining the aggregated patient data set 112.

Example and non-limiting clinical alert value(s) 1406 include: an inconsistent data (e.g., from the EMRs 102 and/or clinically provided data) alert, a health status change alert, a treatment scheduling alert, a treatment planning alert, a treatment result alert, a billing status alert, a resident status alert, and/or a treatment recommendation alert.

In certain embodiments, the reporting circuit 1210 further determines at least one of a target schedule (e.g., availability of personnel, availability of a particular person, and/or availability of one or more facilities such as medical equipment, lab availability, etc.), a target location (e.g., a facility, a portion of a facility such as a floor or a wing, or a group of facilities), and provides the clinical alert value 1206 further in response to the target schedule and/or target location.

An example system 1400 further includes a clinical dashboard circuit 1208 that implements a clinical dashboard 118 having a user interface 1214 accessible to at least one external computing device 1222, where the clinical dashboard circuit 1208 further displays the clinical alert value 1406 on the clinical dashboard 118 (e.g., thereby making the clinical alert value 1406 available to a user 1224 and/or user device 1222). An example clinical dashboard 118 further includes a display 1408, such as an alert display, an intervention recommendation display, a live quality measure display, a resident status display, and/or a time dependent value for any of the foregoing. An example clinical dashboard 118 further includes a patient monitoring interface, such as a patient criteria value 1412 and/or a selected time value 1410, where the clinical dashboard circuit 1208 further displays the clinical alert value 1406 in response to a selection of the patient criteria value 1412 and/or the selected time value 1410. For example, the patient monitoring interface allows the decision making platform 100 and/or the user 1224 to determine patient-based criteria for clinical alerts (e.g., watching a particular patient, list of patients, grouping category of patients, and/or patients having certain diagnoses, quality measure indicators, changes in condition, etc.) and/or time-based criteria for clinical alerts (e.g., watching during a particular period of time—past, present, and/or future, and/or turning off alerts during a particular period of time).

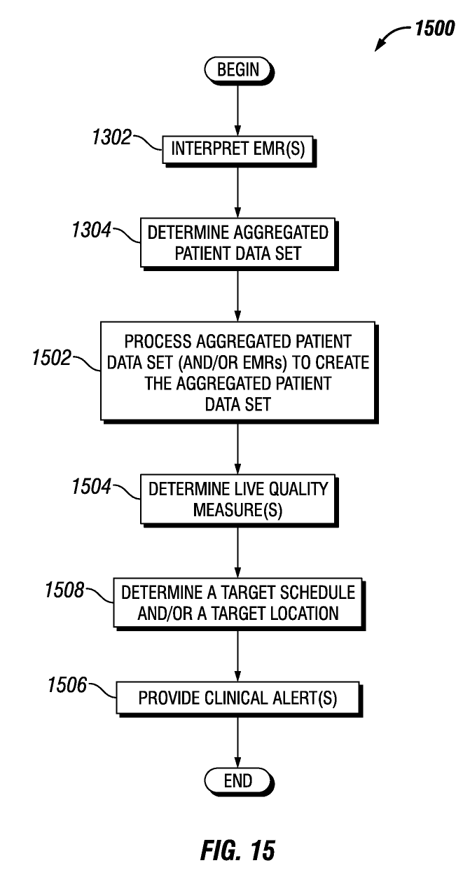

Referencing FIG. 15, a schematic flow diagram depicts an example procedure 1500 for providing a clinical alert value. The example procedure 1500 includes an operation 1302 to interpret a number of EMRs, and an operation 1304 to determine an aggregated patient data set. In certain embodiments, the procedure 1500 includes an operation 1502 to process the aggregated patient data set, for example by standardizing input data, accommodating output data requirements, and/or filtering the aggregated patient data set. The example procedure 1500 further includes an operation 1504 to determine a live quality measure corresponding to a patient in response to the aggregated (and/or filtered) data set. The example procedure 1500 further includes an operation 1506 to provide a clinical alert value(s) in response to the live quality measure. In certain embodiments, the procedure 1500 further includes an operation 1508 to determine a target schedule and/or a target location, where the operation 1506 to provide the clinical alert value is responsive to the target schedule and/or the target location.

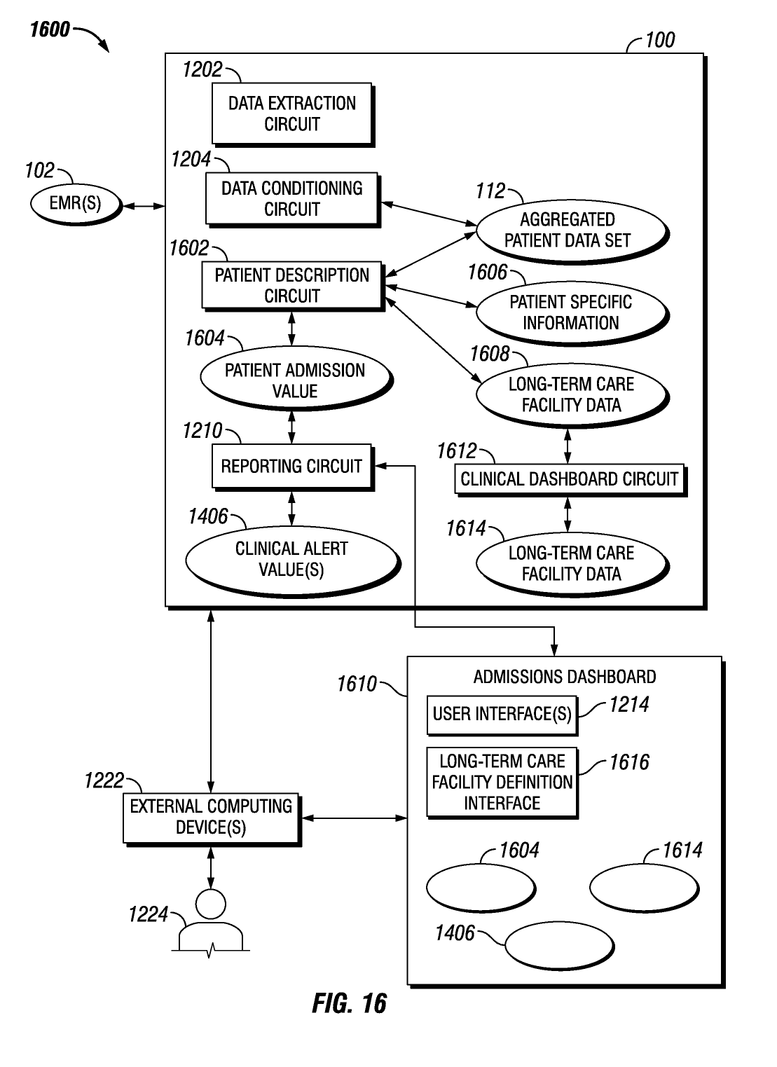

Referencing FIG. 16, an example system 1600 includes a decision making platform 100 having a number of circuits structured to functionally execute certain operations of the system 1600. The example system include a data extraction circuit 1202 that interprets a number of EMRs 102 corresponding to a number of patients from a number of long-term care facilities, and a data conditioning circuit 1204 that determines an aggregated patient data set 112 in response to the number of EMRs 102. The example system 1600 further includes a patient description circuit 1602 that determines a patient admission value 1604 in response to the aggregated patient data set 112. An example patient description circuit 1602 determines the patient admission value 1604 from patient specific information 1606, and from long-term care facility data 1608 corresponding to the patient. Example and non-limiting patient specific information includes diagnoses, treatments, and/or medications of the patient, billing and/or payor information for the patient, readmission data for the patient, and/or ADL, quality measure, and/or scoring data for the patient. Example and non-limiting long-term care facility data corresponding to the patient includes previous long-term care facilities utilized by the patient; staffing, capacity, scores and rankings, and/or equipment information for selected long-term care facilities associated with the patient, and/or patient specific information for other patients at selected long-term care facilities associated with the patient. The patient specific information 1606 and/or long-term care facility data 1608 may be contained within the aggregated patient data set 112, and/or the patient specific information 1606 and/or long-term care facility data 1608 may additionally include outside information, such as information obtained through historical interactions of a health care provider with a long-term care facility, through publicly available information, through regulatory filings, or the like. The example system 1600 further includes a reporting circuit 1210 that implements an admissions dashboard 1610, where the admissions dashboard 1610 includes a user interface 1214 accessible to at least one external computing device 1222, and where the reporting circuit 1210 further displays the patient admission value 1604 on the admissions dashboard 1610.

In certain embodiments, a patient admission value 1604 includes one or more of: a readmission score value corresponding to the patient, a readmission score value corresponding to the long-term care facility data 1608, an EMR 102 access value corresponding to the patient (e.g., where EMR 102 data is available or not available, such as due to the patient not being a patient associated with a facility of the user 1224, and/or where the EMR 102 access corresponding to the patient is either missing and/or unauthorized for access), a previous treatment value corresponding to the patient (e.g., whether the patient has been treated by a facility of the user 1224, and/or any diagnosis, treatments, and/or treatment outcomes), and/or a time dependent value for any of the foregoing (e.g., data for the last 24 hours, last 72 hours, last week, last month, last year, or any other period of interest).

An example system 1600 further includes a long-term facility scoring circuit 1612 that determines a long-term facility performance score 1614 corresponding to at least one of the long-term facilities of the long-term facility data 1608, and where the reporting circuit 1210 further displays the long-term facility performance score on the admissions dashboard 1610. An example system 1600 further includes a long-term care facility definition interface 1616, where the reporting circuit 1210 further displays the long-term care facility performance score 1614 on the admissions dashboard 1610 in response to a selection of the long-term care facility definition interface 1616 (e.g., allowing for the decision making platform 100 and/or the user 1224 to select certain long-term care facilities for consideration, and/or to select geographic areas for consideration with a resulting selection of associated long-term care facilities). In certain embodiments, a long-term facility performance score 1608 includes a readmission rate (e.g., of patients, relevant patients to the patient of the patient specific information 1606, and/or over a selected time period). In certain embodiments, the patient specific information 1606 corresponds to a patient that is currently resident at one of the long-term care facilities under consideration, where the patient description circuit 1602 further predicts the patient admission value 1604 for that patient currently resident at the long-term care facility (e.g., predicting whether and/or when the patient will be re-admitted to a facility of the user 1224, which may be the facility that the patient is currently resident at or another facility, after discharge from the long-term care facility). In certain embodiments, the reporting circuit 1210 further provides a clinical alert value 1406 in response to the patient admission value 1604—for example where the patient admission value 1604 indicates a treatment, intervention, and/or extended stay for the patient may be advisable, and/or where a change in the patient admission value 1604 (e.g., compared to a previous or recent value) indicates that the condition of the patient may be deteriorating or not acceptably improving. An example clinical alert value 1406 includes a treatment follow-up suggestion (e.g., a diagnostic test, treatment regime, treatment schedule change, medication, and/or medication change). Another example clinical alert value 1406 includes a resource allocation value (e.g., a staffing change or requirement, a facility change or requirement, an equipment change or requirement, and/or a time relationship for any of these).

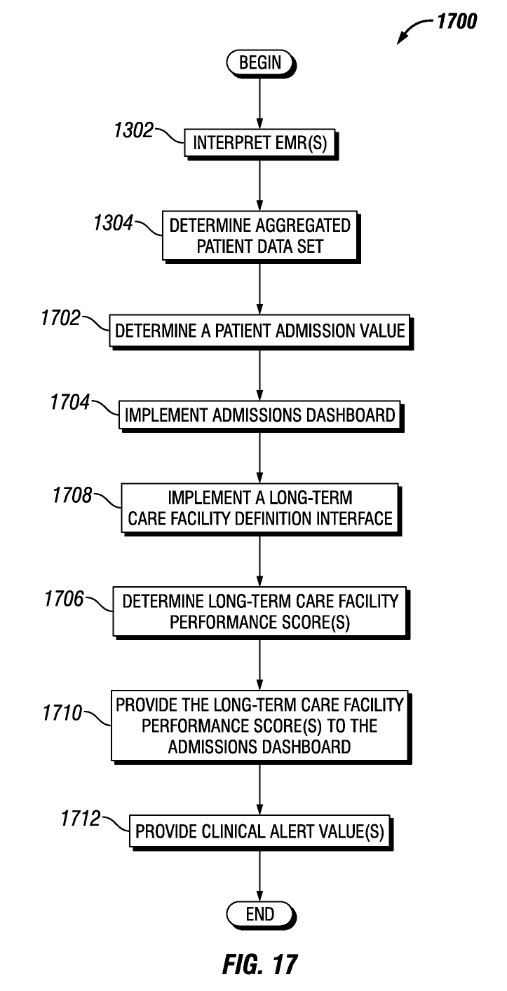

Referencing FIG. 17, a schematic flow diagram depicts an example procedure 1700 for providing a patient admission value. The example procedure 1700 includes an operation 1302 to interpret a number of EMRs corresponding to a number of patients from a number of long-term care facilities, and an operation 1304 to determine an aggregated patient data set in response to the number of EMRs. The example procedure 1700 further includes an operation 1702 to determine a patient admission value in response to the aggregated patient data set. An example operation 1702 includes determining the patient admission value from patient specific information and from long-term care facility data. The example procedure 1700 further includes an operation 1704 to implement an admissions dashboard, where the admissions dashboard includes a user interface accessible to at least one external computing device, and where operation 1704 further includes displaying the patient admission value on the admissions dashboard.

In certain embodiments, the example procedure 1700 further includes an operation 1706 to determine a long-term facility performance score, for example corresponding to at least one of the long-term care facilities of the long-term care facility data. In certain embodiments, the procedure 1700 includes an operation 1708 to implement a long-term care facility definition interface, and where the operation 1706 is performed in response to a selection of the long-term care facility definition interface. In certain embodiments, the example procedure 1700 further includes an operation 1710 to display the long-term facility performance score on the admissions dashboard. An example procedure 1700 further includes an operation 1712 to provide a clinical alert value in response to the patient admission value.

The methods and systems described herein may be deployed in part or in whole through a machine having a computer, computing device, processor, circuit, and/or server that executes computer readable instructions, program codes, instructions, and/or includes hardware configured to functionally execute one or more operations of the methods and systems disclosed herein. The terms computer, computing device, processor, circuit, and/or server, as utilized herein, should be understood broadly.

Any one or more of the terms computer, computing device, processor, circuit, and/or server include a computer of any type, capable to access instructions stored in communication thereto such as upon a non-transient computer readable medium, whereupon the computer performs operations of systems or methods described herein upon executing the instructions. In certain embodiments, such instructions themselves comprise a computer, computing device, processor, circuit, and/or server. Additionally or alternatively, a computer, computing device, processor, circuit, and/or server may be a separate hardware device, one or more computing resources distributed across hardware devices, and/or may include such aspects as logical circuits, embedded circuits, sensors, actuators, input and/or output devices, network and/or communication resources, memory resources of any type, processing resources of any type, and/or hardware devices configured to be responsive to determined conditions to functionally execute one or more operations of systems and methods herein.

Network and/or communication resources include, without limitation, local area network, wide area network, wireless, internet, or any other known communication resources and protocols. Example and non-limiting hardware, computers, computing devices, processors, circuits, and/or servers include, without limitation, a general purpose computer, a server, an embedded computer, a mobile device, a virtual machine, and/or an emulated version of one or more of these. Example and non-limiting hardware, computers, computing devices, processors, circuits, and/or servers may be physical, logical, or virtual. A computer, computing device, processor, circuit, and/or server may be: a distributed resource included as an aspect of several devices; and/or included as an interoperable set of resources to perform described functions of the computer, computing device, processor, circuit, and/or server, such that the distributed resources function together to perform the operations of the computer, computing device, processor, circuit, and/or server. In certain embodiments, each computer, computing device, processor, circuit, and/or server may be on separate hardware, and/or one or more hardware devices may include aspects of more than one computer, computing device, processor, circuit, and/or server, for example as separately executable instructions stored on the hardware device, and/or as logically partitioned aspects of a set of executable instructions, with some aspects of the hardware device comprising a part of a first computer, computing device, processor, circuit, and/or server, and some aspects of the hardware device comprising a part of a second computer, computing device, processor, circuit, and/or server.

A computer, computing device, processor, circuit, and/or server may be part of a server, client, network infrastructure, mobile computing platform, stationary computing platform, or other computing platform. A processor may be any kind of computational or processing device capable of executing program instructions, codes, binary instructions and the like. The processor may be or include a signal processor, digital processor, embedded processor, microprocessor or any variant such as a co-processor (math co-processor, graphic co-processor, communication co-processor and the like) and the like that may directly or indirectly facilitate execution of program code or program instructions stored thereon. In addition, the processor may enable execution of multiple programs, threads, and codes. The threads may be executed simultaneously to enhance the performance of the processor and to facilitate simultaneous operations of the application. By way of implementation, methods, program codes, program instructions and the like described herein may be implemented in one or more threads. The thread may spawn other threads that may have assigned priorities associated with them; the processor may execute these threads based on priority or any other order based on instructions provided in the program code. The processor may include memory that stores methods, codes, instructions and programs as described herein and elsewhere. The processor may access a storage medium through an interface that may store methods, codes, and instructions as described herein and elsewhere. The storage medium associated with the processor for storing methods, programs, codes, program instructions or other type of instructions capable of being executed by the computing or processing device may include but may not be limited to one or more of a CD-ROM, DVD, memory, hard disk, flash drive, RAM, ROM, cache and the like.

A processor may include one or more cores that may enhance speed and performance of a multiprocessor. In embodiments, the process may be a dual core processor, quad core processors, other chip-level multiprocessor and the like that combine two or more independent cores (called a die).

The methods and systems described herein may be deployed in part or in whole through a machine that executes computer readable instructions on a server, client, firewall, gateway, hub, router, or other such computer and/or networking hardware. The computer readable instructions may be associated with a server that may include a file server, print server, domain server, internet server, intranet server and other variants such as secondary server, host server, distributed server and the like. The server may include one or more of memories, processors, computer readable transitory and/or non-transitory media, storage media, ports (physical and virtual), communication devices, and interfaces capable of accessing other servers, clients, machines, and devices through a wired or a wireless medium, and the like. The methods, programs, or codes as described herein and elsewhere may be executed by the server. In addition, other devices required for execution of methods as described in this application may be considered as a part of the infrastructure associated with the server.

The server may provide an interface to other devices including, without limitation, clients, other servers, printers, database servers, print servers, file servers, communication servers, distributed servers, and the like. Additionally, this coupling and/or connection may facilitate remote execution of instructions across the network. The networking of some or all of these devices may facilitate parallel processing of program code, instructions, and/or programs at one or more locations without deviating from the scope of the disclosure. In addition, all the devices attached to the server through an interface may include at least one storage medium capable of storing methods, program code, instructions, and/or programs. A central repository may provide program instructions to be executed on different devices. In this implementation, the remote repository may act as a storage medium for methods, program code, instructions, and/or programs.

The methods, program code, instructions, and/or programs may be associated with a client that may include a file client, print client, domain client, internet client, intranet client and other variants such as secondary client, host client, distributed client and the like. The client may include one or more of memories, processors, computer readable transitory and/or non-transitory media, storage media, ports (physical and virtual), communication devices, and interfaces capable of accessing other clients, servers, machines, and devices through a wired or a wireless medium, and the like. The methods, program code, instructions, and/or programs as described herein and elsewhere may be executed by the client. In addition, other devices utilized for execution of methods as described in this application may be considered as a part of the infrastructure associated with the client.

The client may provide an interface to other devices including, without limitation, servers, other clients, printers, database servers, print servers, file servers, communication servers, distributed servers, and the like. Additionally, this coupling and/or connection may facilitate remote execution of methods, program code, instructions, and/or programs across the network. The networking of some or all of these devices may facilitate parallel processing of methods, program code, instructions, and/or programs at one or more locations without deviating from the scope of the disclosure. In addition, all the devices attached to the client through an interface may include at least one storage medium capable of storing methods, program code, instructions, and/or programs. A central repository may provide program instructions to be executed on different devices. In this implementation, the remote repository may act as a storage medium for methods, program code, instructions, and/or programs.

The methods and systems described herein may be deployed in part or in whole through network infrastructures. The network infrastructure may include elements such as computing devices, servers, routers, hubs, firewalls, clients, personal computers, communication devices, routing devices and other active and passive devices, modules, and/or components as known in the art. The computing and/or non-computing device(s) associated with the network infrastructure may include, apart from other components, a storage medium such as flash memory, buffer, stack, RAM, ROM and the like. The methods, program code, instructions, and/or programs described herein and elsewhere may be executed by one or more of the network infrastructural elements.

The methods, program code, instructions, and/or programs described herein and elsewhere may be implemented on a cellular network having multiple cells. The cellular network may either be frequency division multiple access (FDMA) network or code division multiple access (CDMA) network. The cellular network may include mobile devices, cell sites, base stations, repeaters, antennas, towers, and the like.

The methods, program code, instructions, and/or programs described herein and elsewhere may be implemented on or through mobile devices. The mobile devices may include navigation devices, cell phones, mobile phones, mobile personal digital assistants, laptops, palmtops, netbooks, pagers, electronic books readers, music players, and the like. These mobile devices may include, apart from other components, a storage medium such as a flash memory, buffer, RAM, ROM and one or more computing devices. The computing devices associated with mobile devices may be enabled to execute methods, program code, instructions, and/or programs stored thereon. Alternatively, the mobile devices may be configured to execute instructions in collaboration with other devices. The mobile devices may communicate with base stations interfaced with servers and configured to execute methods, program code, instructions, and/or programs. The mobile devices may communicate on a peer to peer network, mesh network, or other communications network. The methods, program code, instructions, and/or programs may be stored on the storage medium associated with the server and executed by a computing device embedded within the server. The base station may include a computing device and a storage medium. The storage device may store methods, program code, instructions, and/or programs executed by the computing devices associated with the base station.

The methods, program code, instructions, and/or programs may be stored and/or accessed on machine readable transitory and/or non-transitory media that may include: computer components, devices, and recording media that retain digital data used for computing for some interval of time; semiconductor storage known as random access memory (RAM); mass storage typically for more permanent storage, such as optical discs, forms of magnetic storage like hard disks, tapes, drums, cards and other types; processor registers, cache memory, volatile memory, non-volatile memory; optical storage such as CD, DVD; removable media such as flash memory (e.g., USB sticks or keys), floppy disks, magnetic tape, paper tape, punch cards, standalone RAM disks, Zip drives, removable mass storage, off-line, and the like; other computer memory such as dynamic memory, static memory, read/write storage, mutable storage, read only, random access, sequential access, location addressable, file addressable, content addressable, network attached storage, storage area network, bar codes, magnetic ink, and the like.

Certain operations described herein include interpreting, receiving, and/or determining one or more values, parameters, inputs, data, or other information. Operations including interpreting, receiving, and/or determining any value parameter, input, data, and/or other information include, without limitation: receiving data via a user input; receiving data over a network of any type; reading a data value from a memory location in communication with the receiving device; utilizing a default value as a received data value; estimating, calculating, or deriving a data value based on other information available to the receiving device; and/or updating any of these in response to a later received data value. In certain embodiments, a data value may be received by a first operation, and later updated by a second operation, as part of the receiving a data value. For example, when communications are down, intermittent, or interrupted, a first operation to interpret, receive, and/or determine a data value may be performed, and when communications are restored an updated operation to interpret, receive, and/or determine the data value may be performed.

Certain logical groupings of operations herein, for example methods or procedures of the current disclosure, are provided to illustrate aspects of the present disclosure. Operations described herein are schematically described and/or depicted, and operations may be combined, divided, re-ordered, added, or removed in a manner consistent with the disclosure herein. It is understood that the context of an operational description may require an ordering for one or more operations, and/or an order for one or more operations may be explicitly disclosed, but the order of operations should be understood broadly, where any equivalent grouping of operations to provide an equivalent outcome of operations is specifically contemplated herein. For example, if a value is used in one operational step, the determining of the value may be required before that operational step in certain contexts (e.g. where the time delay of data for an operation to achieve a certain effect is important), but may not be required before that operation step in other contexts (e.g. where usage of the value from a previous execution cycle of the operations would be sufficient for those purposes). Accordingly, in certain embodiments an order of operations and grouping of operations as described is explicitly contemplated herein, and in certain embodiments re-ordering, subdivision, and/or different grouping of operations is explicitly contemplated herein.

The methods and systems described herein may transform physical and/or or intangible items from one state to another. The methods and systems described herein may also transform data representing physical and/or intangible items from one state to another.

The elements described and depicted herein, including in flow charts, block diagrams, and/or operational descriptions, depict and/or describe specific example arrangements of elements for purposes of illustration. However, the depicted and/or described elements, the functions thereof, and/or arrangements of these, may be implemented on machines, such as through computer executable transitory and/or non-transitory media having a processor capable of executing program instructions stored thereon, and/or as logical circuits or hardware arrangements. Example arrangements of programming instructions include at least: monolithic structure of instructions; standalone modules of instructions for elements or portions thereof; and/or as modules of instructions that employ external routines, code, services, and so forth; and/or any combination of these, and all such implementations are contemplated to be within the scope of embodiments of the present disclosure Examples of such machines include, without limitation, personal digital assistants, laptops, personal computers, mobile phones, other handheld computing devices, medical equipment, wired or wireless communication devices, transducers, chips, calculators, satellites, tablet PCs, electronic books, gadgets, electronic devices, devices having artificial intelligence, computing devices, networking equipment, servers, routers and the like. Furthermore, the elements described and/or depicted herein, and/or any other logical components, may be implemented on a machine capable of executing program instructions. Thus, while the foregoing flow charts, block diagrams, and/or operational descriptions set forth functional aspects of the disclosed systems, any arrangement of program instructions implementing these functional aspects are contemplated herein. Similarly, it will be appreciated that the various steps identified and described above may be varied, and that the order of steps may be adapted to particular applications of the techniques disclosed herein. Additionally, any steps or operations may be divided and/or combined in any manner providing similar functionality to the described operations. All such variations and modifications are contemplated in the present disclosure. The methods and/or processes described above, and steps thereof, may be implemented in hardware, program code, instructions, and/or programs or any combination of hardware and methods, program code, instructions, and/or programs suitable for a particular application. Example hardware includes a dedicated computing device or specific computing device, a particular aspect or component of a specific computing device, and/or an arrangement of hardware components and/or logical circuits to perform one or more of the operations of a method and/or system. The processes may be implemented in one or more microprocessors, microcontrollers, embedded microcontrollers, programmable digital signal processors or other programmable device, along with internal and/or external memory. The processes may also, or instead, be embodied in an application specific integrated circuit, a programmable gate array, programmable array logic, or any other device or combination of devices that may be configured to process electronic signals. It will further be appreciated that one or more of the processes may be realized as a computer executable code capable of being executed on a machine readable medium.

The computer executable code may be created using a structured programming language such as C, an object oriented programming language such as C#, a browser framework such as Angular, or any other high-level or low-level programming language (including assembly languages, hardware description languages, and database programming languages and technologies) that may be stored, compiled or interpreted to run on one of the above devices, as well as heterogeneous combinations of processors, processor architectures, or combinations of different hardware and computer readable instructions, or any other machine capable of executing program instructions.

Thus, in one aspect, each method described above and combinations thereof may be embodied in computer executable code that, when executing on one or more computing devices, performs the steps thereof. In another aspect, the methods may be embodied in systems that perform the steps thereof, and may be distributed across devices in a number of ways, or all of the functionality may be integrated into a dedicated, standalone device or other hardware. In another aspect, the means for performing the steps associated with the processes described above may include any of the hardware and/or computer readable instructions described above. All such permutations and combinations are contemplated in embodiments of the present disclosure.

What is claimed is:

1. A system for determining readmission risks for patients, the system comprising:
a decision making platform comprising:
a data extraction circuit structured to interpret a plurality of electronic medical records (EMRs);
a data conditioning circuit structured to determine an aggregated patient data set in response to the plurality of EMRs;
a patient readmission prediction circuit structured to determine a readmission risk for a patient in response to the aggregated patient data set; and
a reporting circuit structured to provide a readmission response value in response to the readmission risk for the patient;
a clinical dashboard circuit structured to implement a transitions of care dashboard, the transitions of care dashboard comprising:
a user interface accessible to at least one external computing device;
a time selection interface;
readmission rates corresponding to at least a portion of the aggregated patient data set; and
wherein the clinical dashboard circuit is further structured to display the provided readmission response value on the transitions of care dashboard, and to perform at least one of sorting, grouping, or filtering the transitions of care dashboard in response to a selection of the time selection interface.

2. The system of claim 1, wherein the transitions of care dashboard further comprises a patient selection interface, and wherein the clinical dashboard circuit is further structured to display a plurality of readmission values in response to a patient selection of the patient selection interface.

3. The system of claim 1, wherein the transitions of care dashboard further comprises a plurality of readmission scores corresponding to a plurality of readmission events, wherein the plurality of readmission scores comprise weighted scores.

4. A system, comprising:
a decision making platform comprising:
a data extraction circuit structured to interpret a plurality of electronic medical records (EMRs);
a data conditioning circuit structured to determine an aggregated patient data set in response to the plurality of EMRs;
a patient readmission prediction circuit structured to determine a readmission risk for a patient in response to the aggregated patient data set; and
a reporting circuit structured to provide a readmission response value in response to the readmission risk for the patient.

5. The system of claim 4, wherein the readmission response value comprises at least one of: a treatment recommendation value; a patient alert value; a treatment time value; an intervention recommendation value; and a readmission risk report corresponding to a patient.

6. The system of claim 4, further comprising a clinical dashboard circuit structured to implement a transitions of care dashboard, the transitions of care dashboard comprising a user interface accessible to at least one external computing device, and wherein the transitions of care dashboard further comprises at least one of: a plurality of readmission scores corresponding to a plurality of readmission events; readmission rates corresponding to at least a portion of the aggregated patient data set; a plurality of diagnosis values corresponding to a plurality of readmission events; and a plurality of comorbidity values corresponding to a plurality of readmission events.

7. The system of claim 4, further comprising a clinical dashboard circuit structured to implement a transitions of care dashboard, the transitions of care dashboard comprising a user interface accessible to at least one external computing device and readmission rates corresponding to at least a portion of the aggregated patient data set, and wherein the readmission rates comprise at least one readmission rate selected from the readmission rates consisting of: a 30-day readmission rate value; a monthly readmission rate value; a readmission rate value for a selected time period; and a plurality of readmission rates each corresponding to a selected patient grouping category.

8. The system of claim 7, wherein the transitions of care dashboard further comprises a time selection interface, wherein the clinical dashboard circuit is further structured to perform at least one of sorting, grouping, or filtering the transitions of care dashboard in response to a selection of the time selection interface.

9. The system of claim 8, wherein the transitions of care dashboard further comprises a patient selection interface, and wherein the clinical dashboard circuit is further structured to display a plurality of readmission values in response to a patient selection of the patient selection interface.

10. The system of claim 9, wherein the plurality of readmission values comprise at least one value selected from the values consisting of: active diagnoses corresponding to the patient selection; comorbidities corresponding to the patient selection; a readmission rate value corresponding to the patient selection; a readmission score value corresponding to the patient selection; and a patient alert value history corresponding to the patient selection.

11. The system of claim 4, further comprising a clinical dashboard circuit structured to implement a transitions of care dashboard, the transitions of care dashboard comprising a user interface accessible to at least one external computing device and a plurality of readmission scores corresponding to a plurality of readmission events, wherein the plurality of readmission scores comprise weighted scores.

12. The system of claim 11, wherein the clinical dashboard circuit is further structured to weight the plurality of readmission scores according to at least one criteria selected from the criteria consisting of: a plurality of patient alert values; a plurality of patient alert values corresponding to a predetermined time period; a length of stay description for the aggregated patient data set; a set of diagnoses corresponding to a portion of the aggregated patient data set; a set of comorbidities corresponding to a portion of the aggregated patient data set; and a time dependent value for any of the foregoing.

13. A method, comprising:
interpreting a plurality of electronic medical records (EMRs);
determining an aggregated patient data set in response to the plurality of EMRs;
determining a readmission risk for a patient in response to the aggregated patient data set; and
providing a readmission response value in response to the readmission risk for the patient.

14. The method of claim 13, further comprising implementing a transitions of care dashboard, the transitions of care dashboard comprising a user interface accessible to at least one external computing device, and wherein the transitions of care dashboard further comprises at least one of: a plurality of readmission scores corresponding to a plurality of readmission events; readmission rates corresponding to at least a portion of the aggregated patient data set; a plurality of diagnosis values corresponding to a plurality of readmission events; and a plurality of comorbidity values corresponding to a plurality of readmission events.

15. The method of claim 13, further implementing a transitions of care dashboard, the transitions of care dashboard comprising a user interface accessible to at least one external computing device and readmission rates corresponding to at least a portion of the aggregated patient data set, and wherein the readmission rates comprise at least one readmission rate selected from the readmission rates consisting of: a 30-day readmission rate value; a monthly readmission rate value; a readmission rate value for a selected time period; and a plurality of readmission rates each corresponding to a selected patient grouping category.

16. The method of claim 15, wherein the transitions of care dashboard further comprises a time selection interface, the method further comprising at least one of sorting, grouping, or filtering the transitions of care dashboard in response to a selection of the time selection interface.

17. The method of claim 16, wherein the transitions of care dashboard further comprises a patient selection interface, the method further comprising displaying a plurality of readmission values in response to a patient selection of the patient selection interface.

18. The method of claim 17, wherein the plurality of readmission values comprise at least one value selected from the values consisting of: active diagnoses corresponding to the patient selection; comorbidities corresponding to the patient selection; a readmission rate value corresponding to the patient selection; a readmission score value corresponding to the patient selection; and a patient alert value history corresponding to the patient selection.

19. The method of claim 13, further implementing a transitions of care dashboard, the transitions of care dashboard comprising a user interface accessible to at least one external computing device and a plurality of readmission scores corresponding to a plurality of readmission events, wherein the plurality of readmission scores comprise weighted scores.

20. The method of claim 19, further comprising weighting the plurality of readmission scores according to at least one criteria selected from the criteria consisting of: a plurality of patient alert values; a plurality of patient alert values corresponding to a predetermined time period; a length of stay description for the aggregated patient data set; a set of diagnoses corresponding to a portion of the aggregated patient data set; a set of comorbidities corresponding to a portion of the aggregated patient data set; and a time dependent value for any of the foregoing.
